(19) United States
(12) Patent Application Publication
(10) Pub. No.: US 2026/0289740 A1
(43) Pub. Date: Sep. 24, 2026

(54) SYSTEMS AND METHODS FOR IMAGE MOTION COMPENSATION

(71) Applicant: PURDUE RESEARCH FOUNDATION, West Lafayette, IN (US)

(72) Inventors: Rachel Kathleen Surowiec, West Lafayette, IN (US); Farhan Sadik, West Lafayette, IN (US)

(21) Appl. No.: 19/563,972

(22) Filed: Mar. 11, 2026

Related U.S. Application Data
(60) Provisional application No. 63/770,181, filed on Mar. 11, 2025.

(51) Int. Cl. G06T 5/60; A61B 6/00; A61B 6/03; A61B 6/50; G06T 5/73; G06T 7/00

(52) U.S. Cl. CPC G06T 5/60; A61B 6/03; A61B 6/505; A61B 6/5264; G06T 5/73; G06T 7/0012; G06T 2207/10081; G06T 2207/20081; G06T 2207/20084; G06T 2207/20192; G06T 2207/30008

(57) ABSTRACT

An imaging system can include a computing device being configured to receive a motion corrupted image of a portion of a subject and provide the motion corrupted image to a first machine learning model. The first machine learning model can be trained to correct motion corruption. The first machine learning model having been trained by using a plurality of training image pairs. Each training image pair of the plurality of training image pairs can include a first image and a second image being a motion corrupted version of the first image. The motion corruption in the second image can be simulated. The computing device can be further configured to receive, from the first machine learning model, a corrected version of the motion corrupted image.

### CROSS-REFERENCE TO RELATED APPLICATIONS

This application claims priority to U.S. Patent Application No. 63/770,181 filed Mar. 11, 2026, and entitled, “Sinogram-Based Simulation And Deep Learning-Based Correction Of Motion Artifacts In High Resolution Peripheral Quantitative Computed Tomography (HR-pQCT) Imaging,” which is hereby incorporated by reference in its entirety.

### STATEMENT REGARDING FEDERALLY SPONSORED RESEARCH

This invention was made with government support under P30 AR072581 and LRP 1L30DK130133-0 awarded by the National Institutes of Health. The government has certain rights in the invention.

### BACKGROUND

Medical images, from various imaging modalities, facilitate diagnosing various diseases, conditions, as well as monitoring those conditions. During an image acquisition, however, a patient may move leading to motion artifacts, which can degrade the images. Therefore, it would be desirable to have improved systems and methods for image motion compensation.

### SUMMARY

Rigid-motion artifacts, such as cortical bone streaking and trabecular smearing, hinder in-vivo assessment of bone microstructures in high-resolution peripheral quantitative computed tomography (HR-pQCT). Despite various motion grading techniques, no comprehensive motion correction methods exist due to the lack of standardized degradation models. The present disclosure introduces a sinogram-based method to simulate motion artifacts in HR-pQCT images, creating paired datasets of motion-corrupted images and their corresponding ground truth. This approach establishes a standardized degradation model for motion artifacts, which may be integrated into a supervised learning model for motion correction. As such, the present disclosure describes an Edge-enhanced Self-attention Wasserstein Generative Adversarial Network with Gradient Penalty (“ESWGAN-GP”) to address motion artifacts in both simulated (source) and real-world (target) datasets. The model incorporates edge-enhancing skip connections to preserve trabecular edges and self-attention mechanisms to capture long-range dependencies, facilitating motion correction. A visual geometry group (“VGG”)-based perceptual loss may be used to reconstruct fine micro-structural features. Qualitative evaluation confirms that the motion simulation effectively generates realistic motion-corrupted images. The ESWGAN-GP achieves a mean signal-to-noise ratio (“SNR”) of 26.78, structural similarity index measure (“SSIM”) of 0.81, and visual information fidelity (“VIF”) of 0.76 for the source dataset, with higher values (SNR=29.31, SSIM=0.87, VIF=0.81) for the target dataset. This indicates ESWGAN-GP's ability to correct motion artifacts and restore bone microstructure. The technology described herein using deep learning-based motion correction may reduce patient rescans by up to 10% per week, where rescans present a major challenge for the broader adoption of HR-pQCT.

Some examples of the disclosure provide an imaging system. The imaging system can include a computing device being configured to receive a motion corrupted image of a portion of a subject and provide the motion corrupted image to a first machine learning model. The first machine learning model can be trained to correct motion corruption. The first machine learning model having been trained by using a plurality of training image pairs. Each training image pair of the plurality of training image pairs can include a first image and a second image being a motion corrupted version of the first image. The motion corruption in the second image can be simulated. The computing device can be further configured to receive, from the first machine learning model, a corrected version of the motion corrupted image.

In some examples, a computing device can be further configured to construct each training image pair by receiving a first image and introducing a simulation motion artifact into the first image to generate a second image.

In some examples, a computing device can be further configured to introduce a simulation motion artifact to each second image of each training image pair of a plurality of training image pairs by receiving a sinogram of a first image, shifting one or more lines of the sinogram to generate a shifted sinogram, and generating the second image by reconstructing the shifted sinogram.

In some examples, a first machine learning model can correct at least one of an in plane translation of a portion of the subject, an in plane rotation of the portion of the subject, or a z-axis translation of the portion of the subject.

In some examples, a corrected version of a motion corrupted image can be a motion corrected image. A computing device can be further configured to determine, using the motion corrected image, one or more anatomical measurements of the portion of the subject.

In some examples, a computing device can be further configured to determine a presence or a score of a medical condition, based on one or more anatomical measurements.

In some examples, a motion corrupted image can be a high-resolution peripheral quantitative computed tomography (HR-pQCT) image. One or more anatomical measurements can include a cortical thickness, a trabecular number, or a bone mineral density. A medical condition can be osteoporosis.

In some examples, each first image of a plurality of training image pairs can be a motion free image.

In some examples, a computing device can be further configured to generate each second image of each training image pair of a plurality of training image pairs by introducing a simulated motion artifact into a first image to generate a simulated motion image and providing the simulated motion image to a second machine learning model. The second machine learning model can change a first domain of the simulated motion image into a second domain. The computing device can be further configured to receive the second image from the machine learning model.

In some examples, a second machine learning model can be a style transfer machine learning model or a transformer network.

In some examples, a second machine learning model can include a first pair of generators and a second pair of generators.

In some examples, a second machine learning model can include a first cycle generative adversarial network (CycleGAN) and a second CycleGAN. The first CycleGAN can include a first pair of generators. The second CycleGAN can include the second pair of generators.

In some examples, a motion corruption in each second image of a plurality of training image pairs can be introduced directly into a sinogram of a first image to generate each second image.

Some examples of the disclosure provide a method. The method can include receiving a motion corrupted image of a portion of a subject and correcting the motion corrupted image to generate a motion corrected image by providing the motion corrupted image to a first machine learning model and receiving the motion corrected image from the first machine learning model. The first machine learning model has been trained by using a plurality of training image pairs. Each training image pair can include a first image and a second image. The second image can be a motion corrupted version of the first image.

In some examples, a method can include generating each second image of each training image pair of a plurality of training image pairs by introducing a motion artifact in a first image to generate a simulated motion image, providing the simulated motion image to a second machine learning model, and receiving the second image from the second machine learning model.

In some examples, a second machine learning model can be a transfer machine learning model or a transformer network.

In some examples, a method can include determining, using a motion corrected image, one or more anatomical measurements and determining a presence or a score of a medical condition, based on the one or more anatomical measurements.

In some examples, a motion corrupted image can be a high-resolution peripheral quantitative computed tomography (HR-pQCT) image. One or more anatomical measurements can include a cortical thickness, a trabecular number, or a bone mineral density. A medical condition can be osteoporosis.

Some examples of the disclosure provide a method. The method can include receiving a plurality of training image pairs. Each training image pair of the plurality of training image pairs can include a first image and a second image. The second image can be a motion corrupted version of the first image. The method can include providing the plurality of training image pairs to a first machine learning model, and training the first machine learning model to correct a motion artifact in an image, in response to providing the plurality of training image pairs the first machine learning model.

In some examples, a method can include receiving a plurality of training images. The plurality of training images can include a source image, a motion corrupted version of the source image, and a target image. The source image can be substantially free of motion artifacts, the motion corrupted image can include an artificially introduced motion artifact, and the target image can be a real-world motion artifact image. The source image can be of a source domain and the target image can be of a target domain. The method can include providing the plurality of training images to a second machine learning model and training the second machine learning model to change an inputted image having a source domain into an image having the target domain.

In some examples, a computing device is configured to generate a shifted sinogram by taking a projection vector and adding a rotation angle to the projection vector to generate a shifted line.

In some examples, a computing device is configured to apply a transform to a first image to receive a sinogram of the first image.

Some examples of the disclosure provide a method for correcting motion artifacts in high-resolution peripheral quantitative computed tomography (“HR-pQCT”) images. The method can include receiving a motion-corrupted HR-pQCT image and processing the motion-corrupted HR-pQCT image using a trained generative adversarial network (“GAN”) model. The GAN model can include a generator network including a U-Net architecture with skip connections and an edge enhancement module and a discriminator network including self-attention mechanisms. The method can include generating a motion-corrected HR-pQCT image as output from the generator network and outputting the motion-corrected HR-pQCT image.

In some examples, a GAN model is trained using simulated motion-corrupted images generated by applying random rotations to sinogram data of motion-free images, and corresponding ground truth motion-free images.

In some examples, a motion-corrected HR-pQCT image exhibits reduced motion artifacts and preserves bone microstructural features compared to the motion-corrupted HR-pQCT image.

Some examples of the disclosure provide a method for simulating motion artifacts in high-resolution peripheral quantitative computed tomography (“HR-pQCT”) images. The method can include receiving a motion-free HR-pQCT image, defining a set of rotation angles, constructing a projection vector containing a plurality of projection angles, selecting a random rotation angle from the set of rotation angles, generating a shifted projection vector by adding the random rotation angle to the projection vector, computing a Radon transform using the shifted projection vector to generate a shifted sinogram, selecting a subset of projection signals from the shifted sinogram, substituting the subset of projection signals from the shifted sinogram into corresponding locations in the artifact-free sinogram to generate a motion-corrupted sinogram, and reconstructing a motion-corrupted HR-pQCT image from the motion-corrupted sinogram using an iterative reconstruction technique.

In some examples, a method can include pairing a motion-corrupted HR-pQCT image with a motion-free HR-pQCT image to create a training data pair and using the training data pair to train a deep learning model for correcting motion artifacts in HR-pQCT images. In some examples, the deep learning model can be a GAN model.

### DESCRIPTION OF THE DRAWINGS

FIG. 1 shows a block diagram of an example of image reconstruction system according to some examples described in the present disclosure.

FIG. 2 shows a block diagram of example components that can implement the system of FIG. 1.

FIGS. 3A-B shows an illustration of the proposed ESWGAN-GP network.

FIG. 4 shows a flowchart of a process describing applying data to the trained model.

FIG. 5 shows a flowchart of a process describing the training of the trained model using real scans.

FIG. 6 shows a schematic illustration of a block diagram of an imaging system.

FIG. 7 shows an example of an imaging system, which can be a specific implementation of the imaging system of FIG. 6.

FIG. 8 shows a schematic illustration of a block diagram of the architecture of deep learning models.

FIG. 9 shows a flowchart of a process for simulating motion artifacts in images (e.g., a rigid motion artifact in an image).

FIG. 10 shows a flowchart of a process for training a deep learning model.

FIG. 11 shows a flowchart of a process for training a deep learning model.

FIG. 12 shows a flowchart of a process for correcting a motion artifact (e.g., one or more motion artifacts) in an image (e.g., a medical image).

FIG. 13A shows an illustration of a sinogram of a motion free HR-pQCT image.

FIG. 13B shows an illustration of the k-space of FIG. 13A.

FIG. 13C shows an illustration of the k space of the motion corrupted FIG. 13A.

FIG. 14 shows original images, simulated motion artifact images, and real-world motion artifact images.

FIGS. 15A-B shows graphs comparing the PSNR, SSIM, and VIF values of the four models across four anatomical sites.

FIG. 16A shows images showing a qualitative analysis from an ablation study, highlighting the progression of motion correction achieved through the four evaluated models.

FIG. 16B shows images illustrates a second set of comparative results.

FIG. 16C shows a proposed ESWGAN-GP being compared with GAN-CIRCLE in both the simulated unseen test data and the target unseen test data.

FIG. 17 shows a qualitative evaluation of the robustness of ESWGAN-GP.

FIG. 18 shows a qualitative evaluation of the model generalizability in the unseen test data from the source domain is performed.

FIG. 19 shows a qualitative assessment of segmentation performance on motion-corrected images from the unseen source data generated by WGAN, WGAN-GP, SWGAN-GP, and ESWGAN-GP.

FIG. 20A-C show graphs illustrating the performance of ESWGAN-GP in restoring bone geometry in the unseen source dataset.

FIGS. 21A-C show Bland-Altman plots evaluating the agreement between ground truth and ESWGAN-GP-predicted bone geometry parameters relative to their mean ground truth values.

FIGS. 22A-B show Bland-Altman plots depicting the agreement between ground truth and ESWGAN-GP-predicted values for cortical BMD (“Ct.BMD”) and trabecular BMD (“Tb.BMD”).

FIG. 23A-D show images illustrating an overview of motion artifacts in HR-pQCT scans of the distal radius.

FIG. 24 shows a schematic illustration demonstrating the domain gap between the simulated motion corruption and the target motion corruption.

FIG. 25 shows an architecture of DuCYCADA and SWINDyT:DuCyCADA employs dual generators.

FIG. 26 shows top row displays images of the distal radius, while the bottom row depicts images of the distal tibia.

FIG. 27 shows two T-distributed stochastic neighbor embedding plots.

FIG. 28 shows pairs of motion corrupted images followed by the motion corrected image.

### DETAILED DESCRIPTION

To date, X-ray-based modalities remain the gold standard for non-invasive bone quality measurement and continue to be widely used for the detection of bone fragility and fractures. However, diseases such as osteoporosis, type 2 diabetes, chronic kidney disease (“CKD”), and rheumatoid arthritis alter bone matrix mineralization, resulting in distinct changes to cortical and trabecular microarchitecture. These changes necessitate the use of high-resolution imaging techniques, such as micro-CT, peripheral quantitative computed tomography (“pQCT”), and high-resolution peripheral quantitative computed tomography (“HR-pQCT”), for detailed assessment. HR-pQCT is a form of 3D longitudinal cone beam CT (“CBCT”) imaging that primarily focuses on peripheral skeletal sites such as the distal radius and tibia. It can analyze high-resolution (~61-82 μm isotropic voxel size) micro-architecture of cortical bone, the dense outer layer of long bones, and trabecular bone (the lattice-like structure within the marrow cavity) with nominal radiation (~3 μSv), making it attractive for the pediatric population, patients who need to minimize radiation, for use in clinical trials where imaging multiple time points is desired, among other use cases.

High resolution imaging modalities, including HR-pQCT, are especially helpful for diagnosing or otherwise evaluating the progression of some diseases or conditions, such as osteoporosis, which typically requires imaging modalities that can discern small features (e.g., bone microstructure and mineralization). However, high resolution imaging modalities typically require long acquisition times (~2 minutes depending on scanner generation and settings), which increases the likelihood that a patient moves and introduces motion artifacts. Further, the high-resolution nature of the images can disrupt the small features (e.g., even small motion artifacts can disrupt the micro-structure information of the bone). Current protocols to mitigate subject-specific motion generally includes re-scanning of patients, which may not be feasible in ill patients or patients suffering from tremors, twitches, and spasms, and may not be permissible in a busy clinical workflow. Indeed, in some patient populations, such as pediatric patients, it is difficult if not impossible to coach the patient to keep still. Still further, the relatively long image acquisition of high resolution imaging modalities can make it impossible to completely avoid patient movement, even for the general population.

Motion artifacts, estimated to affect up to 23% of first-generation HR-pQCT scans, are traditionally identified through subjective grading using reference images provided by the manufacturer. This involves scoring the image based on the extent of motion corruption, with a score of 1 indicating the highest image quality and a score of 5 representing the lowest. Specifically, the grading reflects the progression of motion artifacts, ranging from minor horizontal/vertical streaks (score 1) to major streaks (score 5), with disruptions in cortical continuity and trabecular smearing seen in score 4 and up. Previous approaches were the first to propose a quantitative metric for measuring motion artifacts using raw sinogram data. They used image similarity metrics, including the sum of squared differences (“SSD”) and normalized cross-correlation (“NCC”), between two aligned projections at 0° and 180°. The underlying assumption was that motion significantly alters the acquisition mode, magnitude, and timing. Ideally, projections at 0° should match those at 180°, and any discrepancies would indicate motion artifacts. More recently, deep learning-based methods have emerged as effective tools for motion grading in HR-pQCT. For example, previous approaches introduced convolutional neural networks to identify five distinct levels of motion grades, addressing limitations of subjective manual grading and improving study comparability. Even so, subject-specific grading remains the prevalent method among users, and there are currently no available reproducible codes for deep learning-based automated grading systems.

Some estimate that during a typical HR-pQCT scan (e.g., lasting two minutes), nearly 40% of the imaging data (e.g., which can be around 2 gigabytes of imaging data per scan) is unusable due to motion artifacts in the images (e.g., rigid motion artifacts). One possible solution would be to remove the motion artifact imaging data or images, leaving the artifact free images. However, being left with a set of artifact free images to, for example, derive anatomical measurements is insufficient. That is, there is not enough residual imaging data to accurately and confidently derive those anatomical measurements. This, therefore, necessitates rescanning of the patient. But, even aside from workflow constraints (e.g., assuming there is a scanner available), patients may not be able to be rescanned as soon as desired, due to daily (or other time period) radiation dose constraints. Further, even assuming that the patient could be rescanned again, the new imaging data may, again, be corrupted with motion artifacts-potentially because the underlying issue causing the motion artifacts (e.g., a patient's tremor) has not been addressed. Accordingly, addressing motion correction, particularly in this context, is a critical need.

Some examples of the disclosure provide advantages to these issues, and others, by providing improved systems and methods for image motion compensation. For example, some examples of the disclosure provide a process of generating training image pairs, which can be used to train a first machine learning model (e.g., a deep learning model) to correct motion artifacts in subsequent images. Each training image pair of a plurality of training image pairs can be generated as follows. A high quality motion free image is selected (e.g., having a visual grading scale (“VGS”) score that is strong, such as less than or equal to two) and a motion artifact is artificially introduced in this image to create a motion corrupted version of the image. The motion corrupted version of the image, acting as motion version image, along with the motion free variant, acting as a ground truth, form a training image pair. These plurality of training image pairs are provided to the first machine learning model to train the first machine learning model (e.g., a deep learning model) to decrease, remove, mitigate, etc., a motion corruption artifact in a subsequent image. In some examples, artificially introducing motion artifacts (e.g., or otherwise simulating the motion artifacts, such as introducing motion artifacts directly in the sinogram of the image) can be helpful for a number of reasons. First, simulating motion artifacts preserves a corresponding ground truth image (e.g., free of motion artifacts), which together are used to train the machine learning model. This can ensure that features are mapped between images, and the machine learning model can eventually discern motion corruption. This accurate feature correspondence would not occur in two different images (e.g., even if acquired at slightly different times of the same patient) since the features between the images would not entirely correspond. Second, training data, including in the HR-pQCT context, is lacking. In other words, currently there is not enough high quality training pairs to effectively train the first machine learning model.

Some examples of the disclosure also provide a process of improving the quality of the motion corrupted image (e.g., the motion simulated image) in each training pair of the plurality of training pairs. In some cases, motion simulated images or artificially generated motion artifacts may lack the qualities of a true motion artifact (e.g., during an image acquisition). For example, the image quality may lack sharpness, may be blurry, and may not effectively mirror a motion artifact (e.g., cortical smearing). In some cases, the lack of image quality can be due to a domain gap between a simulated motion corrupted image and a true motion corrupted image. In some cases, this gap can be reduced and the image quality of a simulated motion image can be improved with style transfer. For example, a second machine learning model (e.g., a style transfer machine learning model) can be trained to improve the quality of an inputted simulated motion corrupted image. In some cases, the second machine learning model can include a first cycle generative adversarial network (“CycleGAN”) and a second CycleGAN. The first and second CycleGAN can be complementary, in that, the first CycleGAN learns between the simulated motion corrupted images and actual (e.g., real-world) motion corrupted images, while the second CycleGAN enforces feature-level alignment (e.g., directly in the probability space). The first and the second CycleGANs form a Dual Cycle Consistent Adversarial Domain Adaptation (“DuCyCADA”), The DuCyCADA can maintain cycle consistency (e.g., the first CycleGAN) by reducing hallucinations. Additionally, the DuCyCADA can perform adversarial alignment (e.g., the second CycleGAN) to force domain alignment. These improved simulated motion corrupted images can replace the previous simulated motion corrupted image in each training image pair, and these training image pairs can be used to train the first machine learning model (e.g., that mitigates motion corruption in real-world).

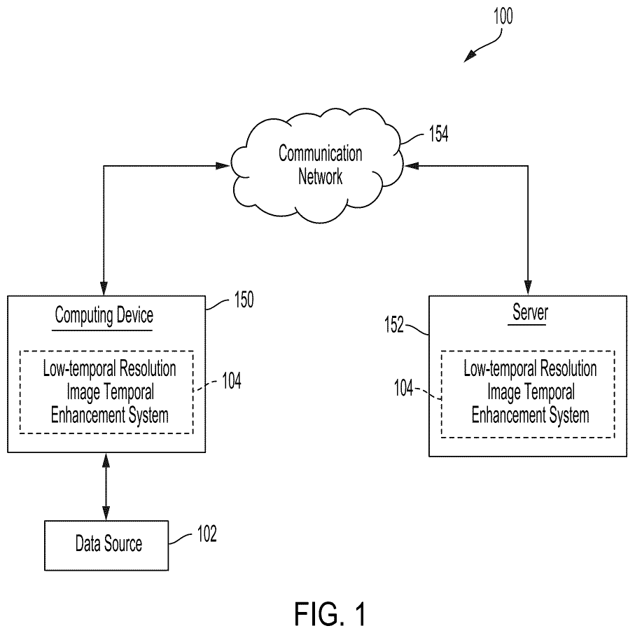

FIG. 1 shows an example of a system 100 for reconstructing medical image data in accordance with some examples described in the present disclosure. As shown in FIG. 1, a computing device 150 can receive one or more types of data (e.g., CT image data) from a data source 102. In some examples, computing device 150 can execute at least a portion of a low-temporal resolution image temporal enhancement system 104 to generate medical image data (e.g., simulated motion corrupted CT image data) from data received from the data source 102.

Additionally, or alternatively, in some examples, the computing device 150 can communicate information about data received from the data source 102 to a server 152 over a communication network 154, which can execute at least a portion of the low-temporal resolution image temporal enhancement system 104. In such examples, the server 152 can return information to the computing device 150 (and/or any other suitable computing device) indicative of an output of the low-temporal resolution image temporal enhancement system 104.

In some examples, computing device 150 and/or server 152 can be any suitable computing device or combination of devices, such as a desktop computer, a laptop computer, a smartphone, a tablet computer, a wearable computer, a server computer, a virtual machine being executed by a physical computing device, and so on. The computing device 150 and/or server 152 can also reconstruct images from the data.

In some examples, data source 102 can be any suitable source of data (e.g., measurement data, images reconstructed from measurement data, processed image data), such as a medical imaging system (e.g., a CT system, a PET system, MRI system, an x-ray, etc.), another computing device (e.g., a server storing measurement data, images reconstructed from measurement data, processed image data), and so on. In some examples, data source 102 can be local to computing device 150. For example, data source 102 can be incorporated with computing device 150 (e.g., computing device 150 can be configured as part of a device for measuring, recording, estimating, acquiring, or otherwise collecting or storing data). As another example, data source 102 can be connected to computing device 150 by a cable, a direct wireless link, and so on. Additionally, or alternatively, in some examples, data source 102 can be located locally and/or remotely from computing device 150, and can communicate data to computing device 150 (and/or server 152) via a communication network (e.g., communication network 154).

In some examples, communication network 154 can be any suitable communication network or combination of communication networks. For example, communication network 154 can include a Wi-Fi network (which can include one or more wireless routers, one or more switches, etc.), a peer-to-peer network (e.g., a Bluetooth network), a cellular network (e.g., a 3G network, a 4G network, etc., complying with any suitable standard, such as CDMA, GSM, LTE, LTE Advanced, WiMAX, etc.), other types of wireless network, a wired network, and so on. In some examples, communication network 154 can be a local area network, a wide area network, a public network (e.g., the Internet), a private or semi-private network (e.g., a corporate or university intranet), any other suitable type of network, or any suitable combination of networks. Communications links shown in FIG. 1 can each be any suitable communications link or combination of communications links, such as wired links, fiber optic links, Wi-Fi links, Bluetooth links, cellular links, and so on.

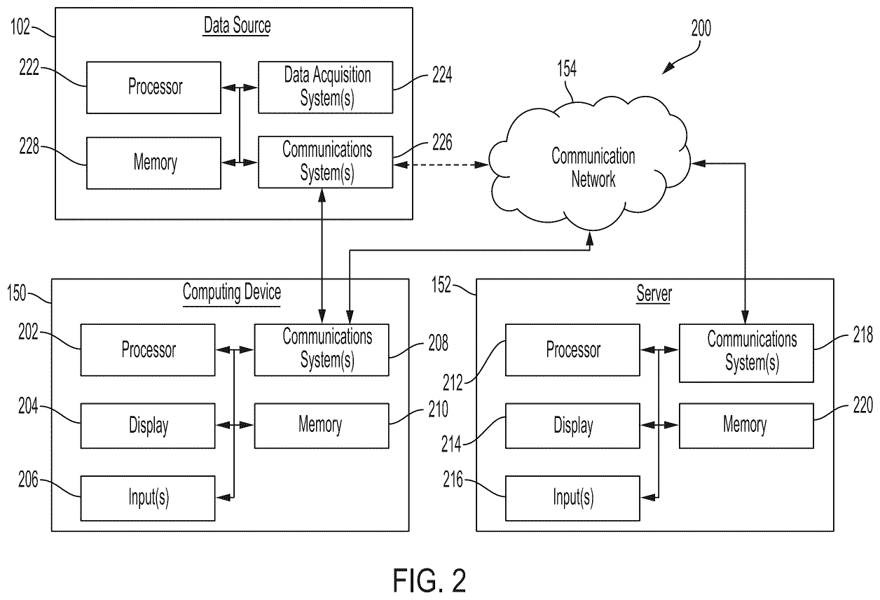

Referring now to FIG. 2, an example of hardware 200 that can be used to implement data source 102, computing device 150, and server 152 in accordance with some examples of the systems and methods described in the present disclosure is shown.

As shown in FIG. 2, in some examples, computing device 150 can include a processor 202, a display 204, one or more inputs 206, one or more communication systems 208, and/or memory 210. In some examples, processor 202 can be any suitable hardware processor or combination of processors, such as a central processing unit (CPU), a graphics processing unit (GPU), and so on. In some examples, display 204 can include any suitable display devices, such as a liquid crystal display (LCD) screen, a light-emitting diode (LED) display, an organic LED (OLED) display, an electrophoretic display (e.g., an “e-ink” display), a computer monitor, a touchscreen, a television, and so on. In some examples, inputs 206 can include any suitable input devices and/or sensors that can be used to receive user input, such as a keyboard, a mouse, a touchscreen, a microphone, and so on.

In some examples, communications systems 208 can include any suitable hardware, firmware, and/or software for communicating information over communication network 154 and/or any other suitable communication networks. For example, communications systems 208 can include one or more transceivers, one or more communication chips and/or chip sets, and so on. In a more particular example, communications systems 208 can include hardware, firmware, and/or software that can be used to establish a Wi-Fi connection, a Bluetooth connection, a cellular connection, an Ethernet connection, and so on.

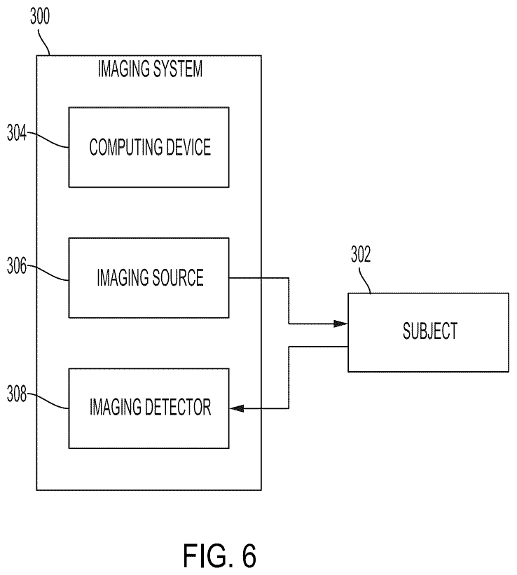

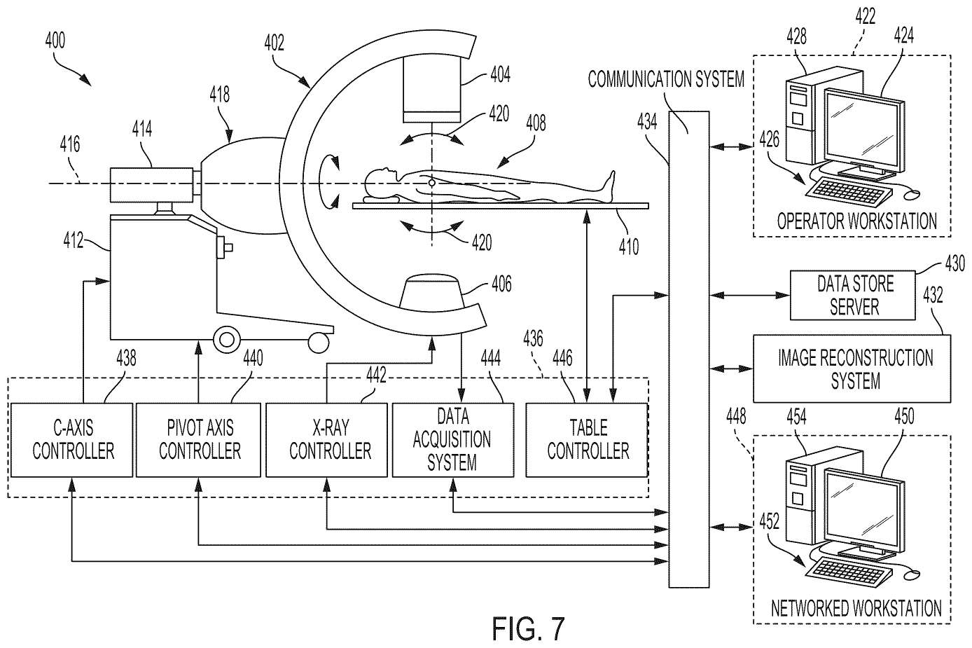

In some examples, memory 210 can include any suitable storage device or devices that can be used to store instructions, values, data, or the like, that can be used, for example, by processor 202 to present content using display 204, to communicate with server 152 via communications system(s) 208, and so on. Memory 210 can include any suitable volatile memory, non-volatile memory, storage, or any suitable combination thereof. For example, memory 210 can include random-access memory (RAM), read-only memory (ROM), electrically programmable ROM (EPROM), electrically erasable ROM (EEPROM), other forms of volatile memory, other forms of non-volatile memory, one or more forms of semi-volatile memory, one or more flash drives, one or more hard disks, one or more solid state drives, one or more optical drives, and so on. In some examples, memory 210 can have encoded thereon, or otherwise stored therein, a computer program for controlling operation of computing device 150. In such examples, processor 202 can execute at least a portion of the computer program to present content (e.g., images, user interfaces, graphics, tables), receive content from server 152, transmit information to server 152, and so on. For example, the processor 202 and the memory 210 can be configured to perform the methods described herein (e.g., the method of FIG. 6, the method of FIG. 7).

In some examples, server 152 can include a processor 212, a display 214, one or more inputs 216, one or more communications systems 218, and/or memory 220. In some examples, processor 212 can be any suitable hardware processor or combination of processors, such as a CPU, a GPU, and so on. In some examples, display 214 can include any suitable display devices, such as an LCD screen, LED display, OLED display, electrophoretic display, a computer monitor, a touchscreen, a television, and so on. In some examples, inputs 216 can include any suitable input devices and/or sensors that can be used to receive user input, such as a keyboard, a mouse, a touchscreen, a microphone, and so on.

In some examples, communications systems 218 can include any suitable hardware, firmware, and/or software for communicating information over communication network 154 and/or any other suitable communication networks. For example, communications systems 218 can include one or more transceivers, one or more communication chips and/or chip sets, and so on. In a more particular example, communications systems 218 can include hardware, firmware, and/or software that can be used to establish a Wi-Fi connection, a Bluetooth connection, a cellular connection, an Ethernet connection, and so on.

In some examples, memory 220 can include any suitable storage device or devices that can be used to store instructions, values, data, or the like, that can be used, for example, by processor 212 to present content using display 214, to communicate with one or more computing devices 150, and so on. Memory 220 can include any suitable volatile memory, non-volatile memory, storage, or any suitable combination thereof. For example, memory 220 can include RAM, ROM, EPROM, EEPROM, other types of volatile memory, other types of non-volatile memory, one or more types of semi-volatile memory, one or more flash drives, one or more hard disks, one or more solid state drives, one or more optical drives, and so on. In some examples, memory 220 can have encoded thereon a server program for controlling operation of server 152. In such examples, processor 212 can execute at least a portion of the server program to transmit information and/or content (e.g., data, images, a user interface) to one or more computing devices 150, receive information and/or content from one or more computing devices 150, receive instructions from one or more devices (e.g., a personal computer, a laptop computer, a tablet computer, a smartphone), and so on.

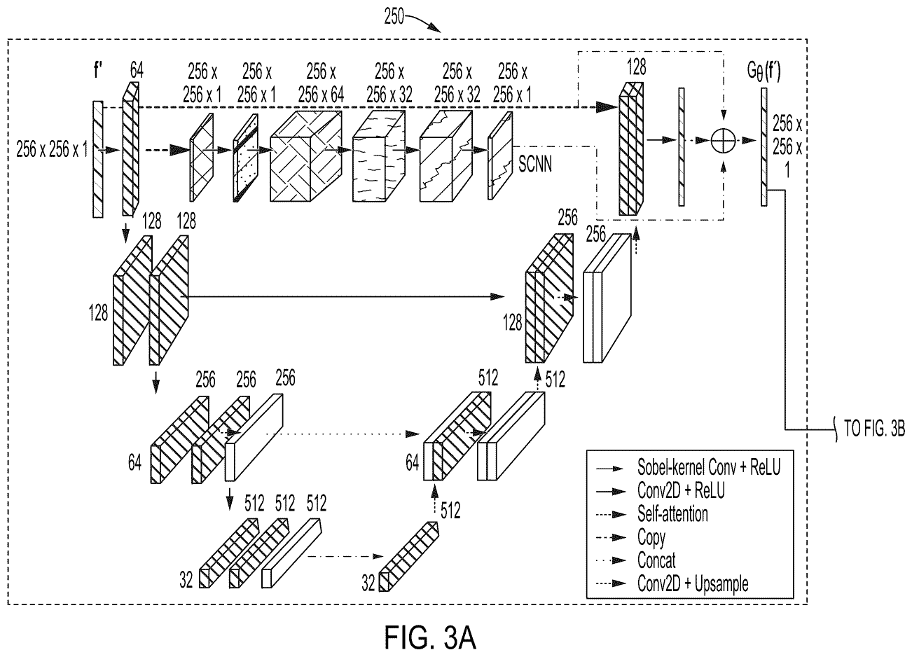

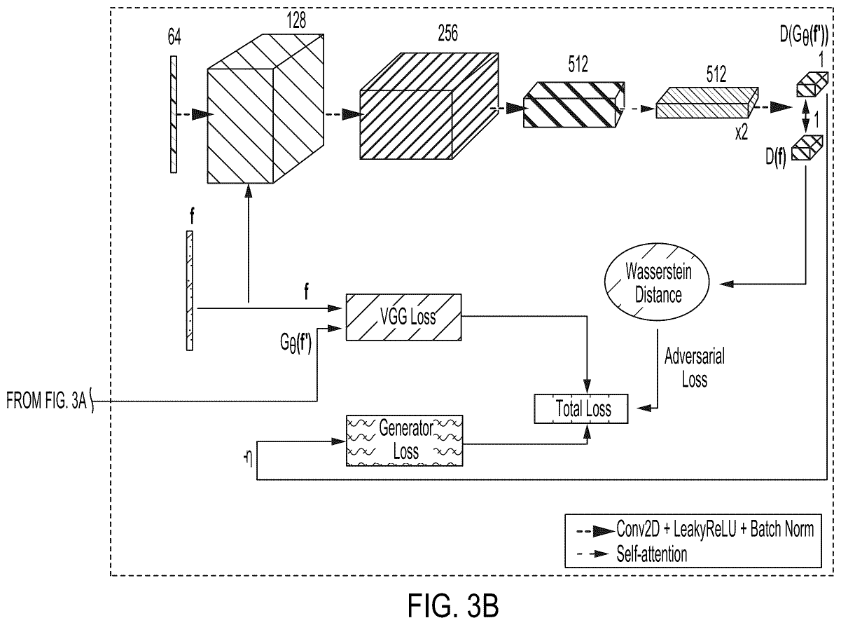

In some examples, the server 152 is configured to perform the methods described in the present disclosure. For example, the processor 212 and memory 220 can be configured to perform the methods described herein (e.g., the method of FIGS. 3A-B).

In some examples, data source 102 can include a processor 222, one or more data acquisition systems 224, one or more communications systems 226, and/or memory 228. In some examples, processor 222 can be any suitable hardware processor or combination of processors, such as a CPU, a GPU, and so on. In some examples, the one or more data acquisition systems 224 are generally configured to acquire data, images, or both, and can include a medical imaging system. Additionally or alternatively, in some examples, the one or more data acquisition systems 224 can include any suitable hardware, firmware, and/or software for coupling to and/or controlling operations of a CT system. In some examples, one or more portions of the data acquisition system(s) 224 can be removable and/or replaceable.

Note that, although not shown, data source 102 can include any suitable inputs and/or outputs. For example, data source 102 can include input devices and/or sensors that can be used to receive user input, such as a keyboard, a mouse, a touchscreen, a microphone, a trackpad, a trackball, and so on. As another example, data source 102 can include any suitable display devices, such as an LCD screen, an LED display, an OLED display, an electrophoretic display, a computer monitor, a touchscreen, a television, etc., one or more speakers, and so on.

In some examples, communications systems 226 can include any suitable hardware, firmware, and/or software for communicating information to computing device 150 (and, in some examples, over communication network 154 and/or any other suitable communication networks). For example, communications systems 226 can include one or more transceivers, one or more communication chips and/or chip sets, and so on. In a more particular example, communications systems 226 can include hardware, firmware, and/or software that can be used to establish a wired connection using any suitable port and/or communication standard (e.g., VGA, DVI video, USB, RS-232, etc.), Wi-Fi connection, a Bluetooth connection, a cellular connection, an Ethernet connection, and so on.

In some examples, memory 228 can include any suitable storage device or devices that can be used to store instructions, values, data, or the like, that can be used, for example, by processor 222 to control the one or more data acquisition systems 224, and/or receive data from the one or more data acquisition systems 224; to generate images from data; present content (e.g., data, images, a user interface) using a display; communicate with one or more computing devices 150; and so on. Memory 228 can include any suitable volatile memory, non-volatile memory, storage, or any suitable combination thereof. For example, memory 228 can include RAM, ROM, EPROM, EEPROM, other types of volatile memory, other types of non-volatile memory, one or more types of semi-volatile memory, one or more flash drives, one or more hard disks, one or more solid state drives, one or more optical drives, and so on. In some examples, memory 228 can have encoded thereon, or otherwise stored therein, a program for controlling operation of data source 102. In such examples, processor 222 can execute at least a portion of the program to generate images, transmit information and/or content (e.g., data, images, a user interface) to one or more computing devices 150, receive information and/or content from one or more computing devices 150, receive instructions from one or more devices (e.g., a personal computer, a laptop computer, a tablet computer, a smartphone, etc.), and so on.

FIGS. 3A-B shows a schematic illustration of an architecture of a machine learning model 250.

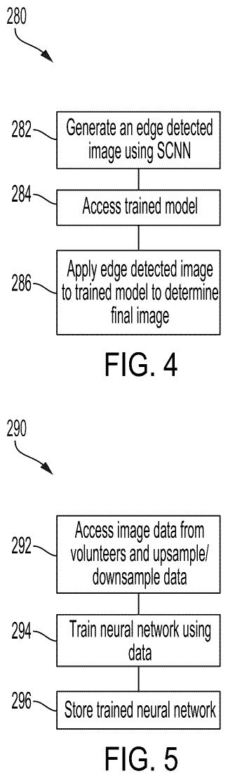

FIG. 4 shows a process 280 for detecting edges in images, while FIG. 5 shows a process 290 for training a neural network. In some cases, a machine learning model (e.g., the machine learning model 250) can include an Edge-enhancer Block: A Sobel-kernel-based Convolutional Neural Network (“SCNN”) module is incorporated along-side the skip connection layer in the uppermost layer of the U-Net-based generator for edge enhancement. In SCNN, the input image undergoes Sobel kernel filtering to generate an edge-detected image 282. This edge-detected image is then subjected to a sequence of three consecutive convolutional layers followed by rectified Linear Units (“ReLU”) before being combined through matrix addition with the final output of the decoder (SCNN in FIGS. 3A-B) 284. To derive the edge image from the input, the Sobel kernel employs two distinct filters oriented along the X and Y directions. Convolution with these filters is followed by calculating the magnitude squared image, which results in the final edge-detected image 286. The rationale for incorporating an edge enhancement module into the deep learning network is grounded in the high-resolution nature of HR-pQCT, which necessitates precise edge reconstruction to ensure measurement of cortical porosity and thickness as well as trabeculae thickness.

In some cases, the machine learning model 250 can include a Self-attention Block. The proposed approach incorporates self-attention blocks into specific layers of the generator and discriminator (white blocks shown in FIGS. 3A-B) to effectively capture long-range dependencies within an image. The attention block, in the generator, is connected directly to the feature maps produced by lower convolutional layers rather than to a flattened output from a fully connected layer, or the upper layers in the generator. This design choice is intentional: by working with 2D feature maps, the attention block can selectively focus on certain regions or patterns within the spatial structure of the image. This setup enables the attention mechanism to emphasize important parts of the image while maintaining the original spatial relationships between pixels, which would be lost if the data were flattened or data were still in the low-level feature state, e.g., in the upper portion of the generator. As a result, the attention block can better highlight relevant features and patterns within the 2D layout of the image.

In some examples, testing data or validation data may be obtained from such a database or from other sources as seen in FIG. 5. For example, subjects (e.g., volunteers) may be scanned during model development to provide testing data to confirm a model built from simulated data is compatible with real scans 292. The data from volunteers can be put into the neural network 294, to further train and be stored in the neural network with real-life scans 296.

FIG. 6 shows a schematic illustration of a block diagram of an imaging system 300. The imaging system 300 can be configured to acquire one or more images of a subject 302 (e.g., a patient). In some cases, the one or more images acquired can be of a portion of the subject 302, such as, for example, a limb. The imaging system 300 can include a computing device 304, an imaging source 306, and an imaging detector 308. The imaging system 300, and specifically, the computing device 304 can cause the imaging source 306 to emit light (e.g., excitation light, x-rays, etc.) towards the subject 302 and receive light in response to the subject 302, which can be received at the imaging detector 308 to acquire an image of the subject 302 (e.g., imaging data that is later reconstructed). In some cases, the imaging source 306 and the imaging detector 308 can be coupled together, such that, movement of the imaging source 306 (e.g., rotation) also moves the imaging detector 308 (and vice versa).

The imaging system 300 can be different imaging modalities, and therefore, the imaging system 300 can be implemented in different ways. For example, the imaging system 300 can be a computed tomography (“CT”) imaging system, a cone beam CT imaging system, a peripheral quantitative CT (“pQCT”) imaging system, a high resolution pQCT (“HR-pQCT”) imaging system, as micro-CT imaging system, an X-ray imaging system, a fluoroscopy imaging system, a positron emission tomography (“PET”) imaging system, optical coherence tomography (“OCT”), optical coherency tomography angiography (“OCTA”), etc.

In some cases, the one or more images acquired by the imaging system 300 can be high resolution images. In some cases, the one or more images acquired by the imaging system 300 can occur during an image acquisition time period. In some cases, the effective spatial resolution can be greater than about 60 μm, less than about 100 μm. In some cases, the effective spatial resolution can be less than about 100 μm, less than about 60 μm, less than about 20 μm (e.g., corresponding to micro-CT), etc. In some cases, the one or more images can be acquired over a period of time greater than one second, two seconds, etc. In some cases, the period of time can be greater than or equal to a corresponding physiological time window (e.g., the duration of a breath, such as during thoracic imaging).

Although FIG. 6 has been described mainly with respect to other medical images (e.g., of a portion of a subject), in other cases, the imaging system 300, and the processes described herein (e.g., to compensate for motion) can be apply to inanimate objects (e.g., a substrate). For example, the imaging system 300 can be an x-ray imaging system to, for example, identify threats in security applications (e.g., airport security). In this case, for example, the images acquired from such an x-ray imaging system can be cleaned of motion artifacts.

FIG. 7 shows an example of an imaging system 400, which can be a specific implementation of the imaging system 300. Accordingly, the description of the imaging system 300 pertains to the imaging system 400 and vice versa. The imaging system 400 can be used with the systems and methods of the present disclosure. The imaging system 400 can be a so-called “C-arm” x-ray imaging system. In other configurations, however, the imaging system 400 can be a fixed-position, a single-source, a bi-plane, or other architecture.

In the example of FIG. 7, the C-arm x-ray imaging system 400 includes a gantry 402 having a C-arm to which an x-ray source assembly 404 is coupled on one end and an x-ray detector array assembly 406 is coupled at its other end. The gantry 402 enables the x-ray source assembly 404 and detector array assembly 406 to be oriented in different positions and angles around a subject 408, such as a medical patient or an object undergoing examination that is positioned on a table 410. When the subject 408 is a medical patient, this configuration enables a physician access to the subject 408.

The x-ray source assembly 404 includes at least one x-ray source that projects an x-ray beam, which may be a fan-beam or cone-beam of x-rays, towards the x-ray detector array assembly 406 on the opposite side of the gantry 402. The x-ray detector array assembly 406 includes at least one x-ray detector, which may include a number of x-ray detector elements. Examples of x-ray detectors that may be included in the x-ray detector array assembly 406 include flat panel detectors, such as so-called “small flat panel” detectors, in which the detector array panel may be around centimeters in size. Such a detector panel allows the coverage of a field-of-view of approximately twelve centimeters.

Together, the x-ray detector elements in the one or more x-ray detectors housed in the x-ray detector array assembly 406 sense the projected x-rays that pass through a subject 408. Each x-ray detector element produces an electrical signal that may represent the intensity of an impinging x-ray beam and, thus, the attenuation of the x-ray beam as it passes through the subject 408. In some configurations, each x-ray detector element is capable of counting the number of x-ray photons that impinge upon the detector. During a scan to acquire x-ray projection data, the gantry 402 and the components mounted thereon rotate about an isocenter of the C-arm x-ray imaging system 400.

The gantry 402 includes a support base 412. A support arm 414 is rotatably fastened to the support base 412 for rotation about a horizontal pivot axis 416. The pivot axis 416 is aligned with the centerline of the table 410 and the support arm 414 extends radially outward from the pivot axis 416 to support a C-arm drive assembly 418 on its outer end. The C-arm gantry 402 is slidably fastened to the drive assembly 418 and is coupled to a drive motor (not shown) that slides the C-arm gantry 402 to revolve it about a C-axis, as indicated by arrows 420. The pivot axis 416 and C-axis are orthogonal and intersect each other at the isocenter of the C-arm x-ray imaging system 400, which is indicated by the black circle and is located above the table 410.

The x-ray source assembly 404 and x-ray detector array assembly 406 extend radially inward to the pivot axis 416 such that the center ray of this x-ray beam passes through the system isocenter. The center ray of the x-ray beam can thus be rotated about the system isocenter around either the pivot axis 416, the C-axis, or both during the acquisition of x-ray attenuation data from a subject 408 placed on the table 410. During a scan, the x-ray source and detector array are rotated about the system isocenter to acquire x-ray attenuation projection data from different angles. By way of example, the detector array is able to acquire thirty projections, or views, per second.

The C-arm x-ray imaging system 400 also includes an operator workstation 422, which typically includes a display 424; one or more input devices 426, such as a keyboard and mouse; and a computer processor 428. The computer processor 428 may include a commercially available programmable machine running a commercially available operating system. The operator workstation 422 provides the operator interface that enables scanning control parameters to be entered into the C-arm x-ray imaging system 400. In general, the operator workstation 422 is in communication with a data store server 430 and an image reconstruction system 432. By way of example, the operator workstation 422, data store sever 430, and image reconstruction system 432 may be connected via a communication system 434, which may include any suitable network connection, whether wired, wireless, or a combination of both. As an example, the communication system 434 may include both proprietary or dedicated networks, as well as open networks, such as the internet.

The operator workstation 422 is also in communication with a control system 436 that controls operation of the C-arm x-ray imaging system 400. The control system 436 generally includes a C-axis controller 438, a pivot axis controller 440, an x-ray controller 442, a data acquisition system (“DAS”) 444, and a table controller 446. The x-ray controller 442 provides power and timing signals to the x-ray source assembly 404, and the table controller 446 is operable to move the table 410 to different positions and orientations within the C-arm x-ray imaging system 400.

The rotation of the gantry 402 to which the x-ray source assembly 404 and the x-ray detector array assembly 406 are coupled is controlled by the C-axis controller 438 and the pivot axis controller 440, which respectively control the rotation of the gantry 402 about the C-axis and the pivot axis 416. In response to motion commands from the operator workstation 422, the C-axis controller 438 and the pivot axis controller 440 provide power to motors in the C-arm x-ray imaging system 400 that produce the rotations about the C-axis and the pivot axis 416, respectively. For example, a program executed by the operator workstation 422 generates motion commands to the C-axis controller 438 and pivot axis controller 440 to move the gantry 402, and thereby the x-ray source assembly 404 and x-ray detector array assembly 406, in a prescribed scan path.

The DAS 444 samples data from the one or more x-ray detectors in the x-ray detector array assembly 406 and converts the data to digital signals for subsequent processing. For instance, digitized x-ray data is communicated from the DAS 444 to the data store server 430. The image reconstruction system 432 then retrieves the x-ray data from the data store server 430 and reconstructs an image therefrom. The image reconstruction system 432 may include a commercially available computer processor, or may be a highly parallel computer architecture, such as a system that includes multiple-core processors and massively parallel, high-density computing devices. Optionally, image reconstruction can also be performed on the processor 428 in the operator workstation 422. Reconstructed images can then be communicated back to the data store server 430 for storage or to the operator workstation 422 to be displayed to the operator or clinician.

The C-arm x-ray imaging system 400 may also include one or more networked workstations 448. By way of example, a networked workstation 448 may include a display 450; one or more input devices 452, such as a keyboard and mouse; and a processor 454. The networked workstation 448 may be located within the same facility as the operator workstation 422, or in a different facility, such as a different healthcare institution or clinic.

The networked workstation 448, whether within the same facility or in a different facility as the operator workstation 422, may gain remote access to the data store server 430, the image reconstruction system 432, or both via the communication system 434. Accordingly, multiple networked workstations 448 may have access to the data store server 430, the image reconstruction system 432, or both. In this manner, x-ray data, reconstructed images, or other data may be exchanged between the data store server 430, the image reconstruction system 432, and the networked workstations 448, such that the data or images may be remotely processed by the networked workstation 448. This data may be exchanged in any suitable format, such as in accordance with the transmission control protocol (“TCP”), the Internet protocol (“IP”), or other known or suitable protocols.

Although the disclosure below will described in reference to the use of a biplane fluoroscopy imaging system (e.g., the “C-arm” x-ray imaging system 400), in other non-limiting examples other imaging systems can be utilized (e.g., a single-plane fluoroscopy imaging system).

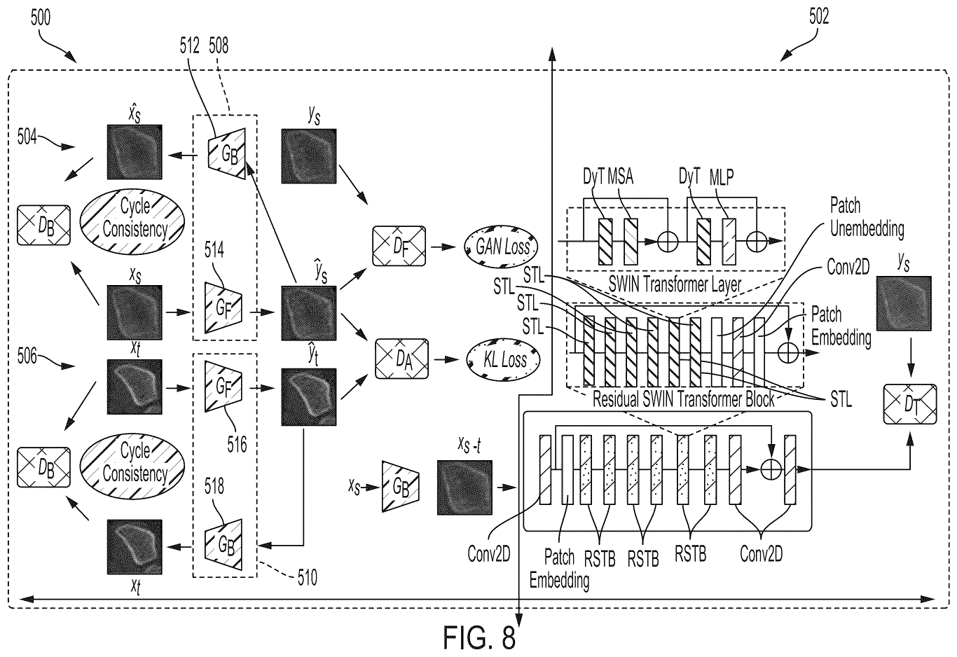

FIG. 8 shows a schematic illustration of a block diagram of the architecture of machine learning models 500, 502. The machine learning model 500 can be trained to improve artificially generated motion artifacts in images. Specifically, the machine learning model 500, for example, when trained, can improve the style (e.g., via style transfer) of the artificially generated motion artifact images. The machine learning model 502, for example, when trained, can reduce motion artifacts in images. In some cases, the machine learning model 502, when trained, can reduce or correct motion-induced artifacts and restore the structural fidelity in the real-world images. The machine learning model 502 can improve the image quality in real-world data (e.g., the overall image quality) by suppressing artifacts caused by the patient a moving object and reconstructing spatially consistent anatomical structures. More specifically, the improvement may include one or more of the following effects: reduction of motion-induced artifacts, including streaking, blurring, distortion, or structural smearing in the image, restoration of fine structural features that may be degraded by the motion improvement in image sharpness and edge definition, improvement in spatial consistency of anatomical structures, enhancement of quantitative image fidelity metrics, such as structural similarity, signal-to-noise ratio, or information fidelity. In some implementations, the machine learning model 502 can also improve the interpretability and reliability of downstream quantitative image analysis, including segmentation, structural measurement, or diagnostic assessment. In some configurations, the machine learning model 502 can perform a domain translation between simulated degraded images and real-world degraded images. For example, the machine learning model 502 can map from a simulated motion-corrupted image domain to a real-world motion-corrupted image domain, or translate synthetically generated artifact images to images that more closely approximate real-world motion corrupted acquisitions. This transformation may be implemented through adversarial learning, generative modeling, style transfer, or other machine learning approaches.

The machine learning model 500 can be a style transfer machine learning model, which can improve the style of an artificially generated motion artifact image (e.g., where the motion artifact is a rigid motion artifact) to more closely resemble an image having a true motion artifact (e.g., a real-world motion artifact, which can be a rigid motion artifact). The machine learning model 500 can include a plurality of generators. For example, the machine learning model 500 can include CycleGANs 504, 506. Each CycleGANs 504, 506 can include respective pair of generators 508, 510. For example, the CycleGAN 504 can include generators 512, 514, while the CycleGAN 506 can include generators 516, 518. Each generator 514, 516 is a forward generator, in which the generator 514 receives a source image (xs) that is the simulated motion corrupted image (e.g., the artificially induced motion corrupted image) and outputs a predicted motion-corrected image (§ s). This predicted motion-corrected image (§ s) and the ground truth image (ys), that is, the image before artificial motion artifacts were introduced, are provided to a discriminator (DF) to minimize GAN loss (and ensure adversarial supervision). The predicted motion-corrected image (§ s) is provided to the generator 512 (e.g., a backward generator) which outputs an inverse reconstructed image of the predicted motion-corrected image (\hat{x}s). The source image (xs) and the inverse reconstructed image (\hat{x}s) are provided to a discriminator (DB) to preserve structural fidelity in the first cycle. The generator 516 receives a target image (xt) that is a true motion corrupted image (e.g., a real-world motion corrupted image, a non-simulated motion corrupted image, etc.) and outputs a motion-corrected image (ft). The motion-corrected image (ft) (e.g., real world motion corrected image) and the predicted motion-corrected image (§ s) are provided to a discriminator (DA) to enforce feature-level alignment though divergence minimization. The motion-corrected image (ft) is proved to the generator 518 (e.g., a backward generator) which outputs an inverse reconstructed image of the motion-corrected image (xt). The target image and the inverse reconstructed image (ît) are provided to a discriminator (DB) to preserve structural fidelity in the second cycle.

In some cases, the machine learning model 500 is trained by performing unsupervised training. For example, a plurality of ground truth images, a plurality of target images, and a plurality of artificially induced motion artifact images can be used to train the machine learning model 500. When the machine learning model 500 is trained, the machine learning model 500 can improve training image pairs. For example, a given training image pair can include a ground truth image, and a modified ground truth image that has artificially induced motion artifacts. The machine learning model 500 can receive a source image (e.g., a motion corrupted image that has artificially introduced motion artifacts) and can output an improved version of the source image (e.g., the motion corrupted image). The improved version of the source image can be a motion corrupted image that has been translated from the source domain to the target domain. In some cases, the source image can be provided to the generator 518 (e.g., the backward generator) and the output from the generator 518 can be the improved version of the source image. This improved source image can replace the previous source image in the given training image pair. This process can improve all training images.

The machine learning model 502 can be trained using the plurality of training image pairs, specifically those that have the improved version of the source image. Accordingly, the machine learning model 502 can be a trained by performing supervised training. Once the machine learning model 502 is trained, the machine learning model 502 can receive a true motion corrupted image (e.g., a real-world motion corrupted image) and output a corrected image. In some cases, the Dynamic Tanh Block (e.g., denoted “DYT” in FIG. 8) can reduce the parameter count, can increase inference speed, while providing superior reconstruction performance as compared to conventional layer normalization (e.g., Shifted Window Transformer).

In some cases, the machine learning model 502, after being trained, can correct a motion artifact inputted into the machine learning model 502. In some cases, the motion artifact can be an in plane translation of a portion of the subject, an in plane rotation of the portion of the subject, or a z-axis translation of the portion of the subject.

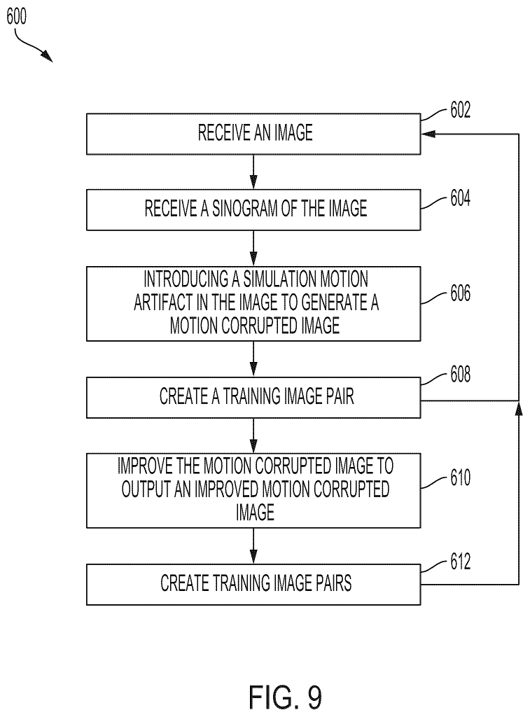

FIG. 9 shows a flowchart of a process 600 for simulating motion artifacts in images (e.g., a rigid motion artifact in an image). In some cases, this process 600 can include generating training image pairs, for example, for training a machine learning model (e.g., the machine learning model 502). The process 600 can be implemented using any of the systems described herein. Further, the process 600 can be implemented using one or more computing devices, as appropriate, and therefore, the process 600 can be a computer implemented method.

At block 602, the process 600 can include receiving an image. In some cases, the image can be acquired, previously, from an imaging system (e.g., the imaging system 300). The image can be a high resolution image. For example, in some examples, high-resolution images may include images acquired using imaging modalities capable of micrometer- to sub-millimeter-scale spatial resolution, including but not limited to high-resolution peripheral quantitative computed tomography (“HR-pQCT”), high-resolution computed tomography (“CT”), fluoroscopy systems, magnetic resonance imaging (“MRI”), or other imaging systems. In some cases, high-resolution can refer to an image having a sufficient spatial resolution to resolve fine structural fine structural or anatomical features of a subject, object, etc., In some cases, this can include the trabecular bone microstructure. High-resolution can also be characterized by a relatively small pixel, voxel dimensions, a high spatial sampling rate, or an imaging acquisition that preserves fine structural detail relative to standard clinical imaging methods. For example, high-resolution images may include images with voxel or pixel sizes sufficiently small to visualize microstructural features, such as trabecular or cortical structures in bone, fine tissue boundaries, or other small-scale anatomical features. In some cases the image can be of a portion of a subject (e.g., a limb of the subject). The image can be a medical image, which can be acquired from various imaging modalities. In some cases, the image can have a VGS score that is less than or equal to two (e.g., two or one).

At block 604, the process 600 can include receiving a sinogram of the image. In some cases, this can include converting the image into a sinogram. For example, this can include applying a Radon transform to the image to generate the sinogram. In some case, the block 602 can be omitted, for example, when images have not been reconstructed from sinograms. For example, the raw sinogram from an imaging system can be provided at the block 604.

At block 606, the process 600 can include introducing a motion artifact (e.g., a simulation motion artifact) in the image to generate a motion corrupted image (e.g., artificially introducing the motion artifact into the image). In some cases, this can include introducing the simulation motion artifact directly into the sinogram of the image. For example, this can include changing the sinogram to introduce the motion artifact. As a more specific example, one or more lines of the sinogram can be shifted to generate a shifted sinogram. In some cases, the shifting of a line can be implemented by taking a projection vector and adding a rotation angle to the projection vector to generate a shifted line. In some configurations, the rotation angle can be in a range from substantially-9 degrees to substantially 5 degrees, −1.5 degrees to 5 degrees, −9 degrees to 1.5 degrees, etc. In some cases, being in a range between −9 degrees to 1.5 degrees can align more closely with plausible motion scenarios (e.g., for a limb). In some cases, a number of the lines can be randomly selected, and each selected line can be shifted by, using the approach above. This random selection of lines can increase the randomness of the motion, as opposed to other approaches, where all the lines are shifted (e.g., when shifting by an angle in the k-space) and which may not mirror true motion artifacts (e.g., since they are not random in the k-space shifting case). Accordingly, in some cases, the shifted sinogram can include one or more modified lines (e.g., a shifted line) and one or more unmodified lines. In this way, not all the lines in the shifted sinogram are actually shifted. In some configurations, once the sinogram has been shifted, the shifted sinogram can be reconstructed to generate the motion corrupted image (e.g., the inverse Rodon transform).

At block 608, the process 600 can include creating training image pairs. For example, a training image pair can include the image (e.g., from the block 602) as a ground truth image and the motion corrupted image (e.g., from the block 606) as the artificially induced motion corruption image.

At block 610, the process 600 can include improving the motion corrupted image (e.g., the motion corrupted image at the block 606) to output an improved motion corrupted image. In some cases, this can include translating the motion corrupted image from a first style (e.g., of the motion corrupted image) to a second style. In other words, this can include adapting a first domain of the motion corrupted image to a second domain, where the second domain is true motion corrupted images. Therefore, in some cases, the improved motion corrupted image can be a domain adapted motion corrupted image. For example, this can include applying a style transfer to the motion corrupted image to generate the improved motion corrupted image. In some cases, this can include providing the motion corrupted image to a style transfer machine learning model (e.g., the machine learning model 500, a portion thereof, etc.) to output the improved motion corrupted image.

At block 612, the process 600 can include creating a training image pair. In some cases, this can include updating the training image pair (e.g., from the block 608). For example, the improved motion corrupted image can be replaced with the motion corrupted image, such that the training image pair includes the image at the block 602 (e.g., the source image, the motion free image, the ground truth image, etc.) and the improved motion corrupted image.

In some cases, the process 600 can be implemented a plurality of times to generate a plurality of training image pairs.

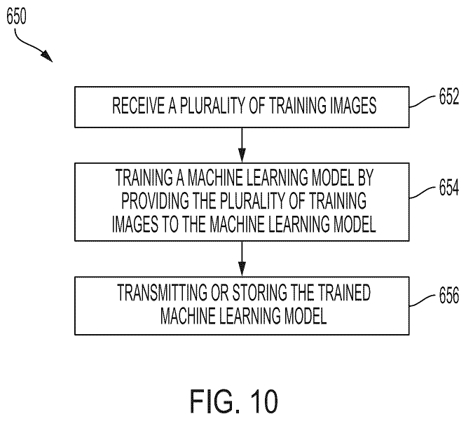

FIG. 10 shows a flowchart of a process 650 for training a machine learning model (e.g., a transfer machine learning model, such as the machine learning model 500). In some cases, this process 650 can be implemented using any of the systems described herein. Further, the process 650 can be implemented using one or more computing devices, as appropriate, and therefore, the process 650 can be a computer implemented method.

At block 652, the process 650 can include receive a plurality of training images. The plurality of images can include one or more source images (e.g., ground truth images, a motion free image, where motion free can be images that have one or more motion artifacts below a specific severity threshold), one or more motion corrupted images (e.g., that have an artificially induced motion artifact), and one or more target images (e.g., images that have actual motion artifacts, real world motion artifacts).

At block 654, the process 650 can include training a machine learning model by providing the plurality of images to the machine learning model. In some cases, training the machine learning model can include implementing unsupervised training of the machine learning model. In some cases, once trained, the trained machine learning model can adapt an inputted image to a domain. For example, an inputted image can have a first domain and the outputted image can have a second domain (e.g., where the second domain is different than the first domain). In some cases, the first domain represents simulated motion-corrupted data, while the second domain consists of real-world motion-corrupted data. Generally, in image reconstruction, once artifacts are introduced by manipulating the raw data to simulate motion, the resulting images rarely return exactly to the original distribution. This process creates a domain gap between the simulated corrupted data and real-world corrupted data. This block 654 provides a mechanism to reduce that domain gap, enabling a machine learning model to be trained on paired data that more closely resembles real-world conditions.

At block 656, the process 650 can include transmitting or storing the trained machine learning model. In some cases, this can include storing the trained machine learning model in memory of a computing device.

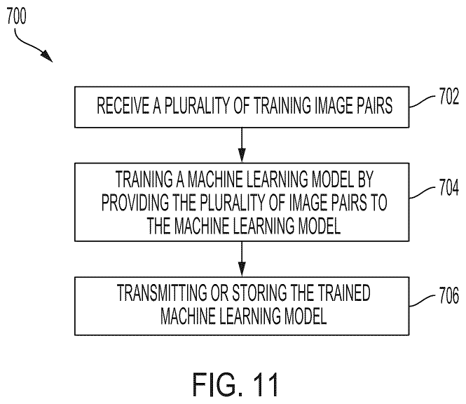

FIG. 11 shows a flowchart of a process 700 for training a machine learning model (e.g., a machine learning model that, when trained, corrects motion artifacts, such as the machine learning model 502). In some cases, this process 700 can be implemented using any of the systems described herein. Further, the process 700 can be implemented using one or more computing devices, as appropriate, and therefore, the process 700 can be a computer implemented method.

At block 702, the process 700 can include receive a plurality of training image pairs. Each training image pair can include a first image and a second image. The first image can be a motion free image, a ground truth image, an image substantially free of motion artifacts (e.g., a VGS score of two or less), etc. The second image can be a motion corrupted version of the first image (e.g., where the second image includes an artificially induced or simulated motion artifact). In some cases, the second image can be an improved version of the motion corrupted version of the first image (e.g., by using the machine learning model 500). For example, the improved version of the motion corrupted version of the first image can have a domain or a style that is different the domain or the style of the motion corrupted version of the first image.

At block 704, the process 700 can include training a machine learning model (e.g., the machine learning model 500) by providing the plurality training image pairs to the machine learning model. In some cases, training the machine learning model can include implementing supervised training of the machine learning model. Fo example, supervised training can include providing the plurality of training image pairs to the machine learning model, where each training image pair includes a first image (e.g., a ground truth image, a motion free image) and a second image (e.g., a motion corrupted version of the first image). The machine learning model can process the second image to generate a predicted motion corrected image. A loss function can be computed by comparing the predicted motion corrected image to the first image of the training image pair. The loss can be back-propagated through the machine learning model to update network parameters (e.g., weights and biases) of the machine learning model. In some cases, an optimizer can be used in conjunction with the loss function to update the network parameters based on computed gradients. The training process can be repeated iteratively over the plurality of training image pairs until the loss function converges, until a predetermined number of training epochs have been completed, or until the machine learning model achieves a desired performance metric (e.g., a target peak signal-to-noise ratio, a target structural similarity index measure, a target visual information fidelity). In some cases, once trained, the trained machine learning model can decrease, remove, mitigate, etc., a motion artifact from an inputted image to the machine learning model. For example, the outputted image can be a motion corrected version of the inputted image.

At block 706, the process 700 can include transmitting or storing the trained machine learning model. In some cases, this can include storing the trained machine learning model in memory of a computing device.

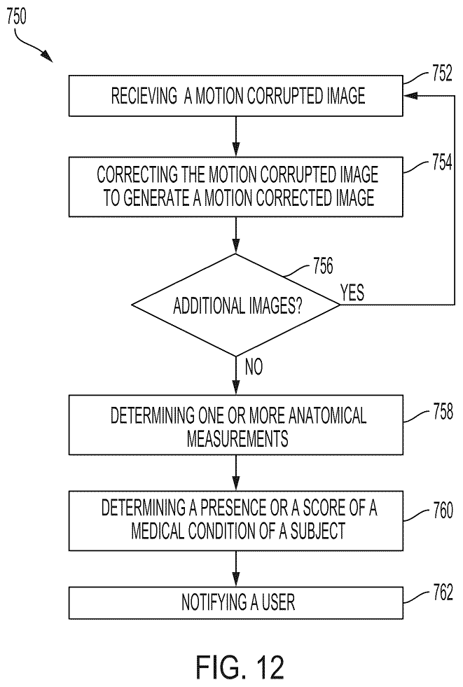

FIG. 12 shows a flowchart of a process 750 for correcting a motion artifact (e.g., one or more motion artifacts) in an image (e.g., a medical image). In some cases, this process 750 can be implemented using any of the systems described herein. For example, the process 750 can be implemented using an imaging system (e.g., the imaging system 300, the imaging system 400, etc.). In this way, the motion corruption can be corrected before transmitting the images for further analysis (e.g., at a computing device), such as, for example, determining one or more anatomical measurements (e.g., of the portion of the subject). Accordingly, the imaging system, in some cases, can implement motion correction at the point of care. In other cases, the process 750 can be implementing using a remote computing device, such as, for example, a server (e.g., the server 152). In this way, motion correction can occur after an image acquisition (e.g., the entire image acquisition from a subject) has been completed. In some cases, the process 750 can be implemented using one or more computing devices, as appropriate, and therefore, the process 750 can be a computer implemented method.

At block 752, the process 750 can include receiving a motion corrupted image. In some cases, this can include acquiring the motion corrupted image using an imaging system. Further, in some cases, this can include generating the motion corrupted image by reconstructing acquired imaging data (e.g., acquired from the imaging system). In some cases, the motion corrupted image can have one or more motion artifacts. In some cases, a motion artifact can be a rigid motion artifact, such as, for example, a blurring, a ghosting, a streak, a smear, etc. In some cases, a motion artifact can be a cortical bone streak, a trabecular smear, etc. In some cases, the motion corrupted image can be a high resolution image. In some cases, the high resolution image can be a HR-pQCT image.

At block 754, the process 750 can include correcting the motion corrupted image to generate a motion corrected image. In some cases, this can include decreasing the motion artifact of the motion corrupted image, mitigating the motion artifact of the motion corrupted image, removing the motion artifact of the motion corrupted image (e.g., completely removing the motion artifact of the motion corrupted image). In some cases, including when the motion corrupted image has a plurality of motion artifacts, the block 754 can include correcting one or more of the motion artifacts. In some cases, the block 754 can include providing the motion corrupted image to a trained machine learning model (e.g., the machine learning model 502 after having been trained)

At block 756, the process 750 can include determining whether there are additional images to be corrected. If at block 756, it is determined that there are additional images to be corrected, the process 750 can proceed back to the block 752 to receive a motion corrupted image (e.g., another motion corrupted image). If, however, at block 756 it is determined that there are no additional images to be corrected, the process 750 can proceed to the block 758.

At block 758, the process 750 can include determining one or more anatomical measurements from at least one of the motion corrected images. For example, an anatomical measurement can be a cortical thickness, a trabecular number, a bone mineral density. In some cases, the block 758 can include determining the one or more anatomical measurements for each motion corrected image for a plurality of motion corrected images (e.g., after a plurality of motion corrected images have been corrected). In some cases, an anatomical measurement can include combining a first anatomical measurement from a first motion corrected image and a second anatomical measurements from a second motion corrupted image. For example, a first cortical thickness can be determined from a first motion corrected image, a second cortical thickness can be determined from a second motion corrected image, and the first cortical thickness and the second cortical thickness can be combined (e.g., averaged) to determine a cortical thickness of a bone of a subject (e.g., a combined cortical thickness).

In some cases, the block 758 can include generating a 3D model from one or more motion corrected images (and uncorrupted images, such as images deemed substantially motion free). In some configurations, therefore, the 3D model can be used to determine the one or more anatomical measurements. For example, the corrected image stack can be used to generate the 3D model of the anatomical structure (e.g., a bone or limb segment) or other feature, from which structural measurements such as cortical thickness, trabecular number, bone mineral density, and other morphometric parameters can be calculated. In some examples, the 3D model can also be used for finite element analysis (“FEA”) or other computational modeling approaches to estimate biomechanical properties, such as stiffness, strength, or fracture risk. The motion correction improves the fidelity of the reconstructed microstructure, which in turn improves the accuracy and reliability of these derived anatomical measurements and biomechanical simulations. In some cases, the generation of the 3D model can be performed using one or more motion-corrected image slices, or by combining measurements derived from multiple motion-corrected images acquired from different regions or time points.

At block 760, the process 750 can include determining a presence or a score of a condition of a subject from the one or more anatomical measurements (e.g., determined at the block 758). For example, the medical condition can be osteoporosis and the presence can be a presence of osteoporosis, while the score can be a severity of the osteoporosis.

At block 762, the process 750 can include notifying a user, for example, of the one or more anatomical measurements, the presence of the medial condition, the score of the medical condition (e.g., a severity of the medical condition), etc. In some cases, this can include generating a report that includes the one or more anatomical measurements, the presence of the medical condition, the score of the medical condition, etc. In some cases, this can include presenting on a display (e.g., of a computing device) the one or more anatomical measurements, the presence of the medical condition, the score of the medical condition, etc.

### EXAMPLES

The following examples have been presented in order to further illustrate aspects of the disclosure, and are not meant to limit the scope of the disclosure in any way. The examples below are intended to be examples of the present disclosure and these (and other aspects of the disclosure) are not to be bounded by theory.

### Example 1

The current work introduces a novel sinogram-based patient motion model for peripheral sites (e.g., tibia, radius) capable of simulating realistic HR-pQCT rigid motion artifacts from artifact-free motion score 1 data. These simulations are paired and used to train an Edge-enhanced Self-attention Wasserstein Generative Adversarial Network with gradient penalty (ESWGAN-GP) to address motion artifacts. Example contributions of this work are outlined below:
1) A patient motion model is introduced, which is based on the rotation of the 2D object to be imaged. The theoretical foundation demonstrates how random alterations to the motion-free sinogram, followed by the reconstruction of the sinogram utilizing Simultaneous Iterative Reconstruction Technique (“SIRT”), result in a corrupted image that mimics real-world motion-corrupted data.
2) Followed by the generation of motion-corrupted and ground truth image pairs, a motion correction method is proposed based on utilizing a Wasserstein Generative Adversarial Network with gradient penalty (“WGAN-GP”) as the backbone network.
3) Self-attention networks in both the generator and the discriminator are utilized to capture the wide range of spatial features (“SWGAN-GP”).
4) A Sobel-kernel-based Convolutional Neural Network (“SCNN”) is integrated in addition to the skip connections in the U-Net generator to enhance the preservation of edges in the bone micro-structures (“ESWGAN-GP”).
5) Utilization of content loss in conjunction with the adversarial loss to ensure robust reconstruction of the bone micro-structures.
   This collective approach constitutes a first-of-its-kind motion correction pipeline specifically designed for solving rigid motion artifacts in HR-pQCT bone imaging.

### Problem Formulation

In this section, the motion correction problem is conceptualized as both a motion correction and a de-blurring problem. Let f∈ [symbol] ^{N}^{_{w}}^{×N}^{ₕ} denotes the object to be imaged which has a width and height of N_{w} and Nₕ. The corresponding sinogram can be represented by s∈ [symbol] ^{N}^{_{ρ}}^{×N}^{_{θ}}, where N_{ρ} is the number of projection lines and N_{θ} is the number of projection angles used while imaging the object. The HR-pQCT acquisition can be described by the following objective function shown in Equation 1.

$$s = ℛ f + ε \qquad ( 1 )$$

Here, [symbol] : [symbol] ^{N}^{_{w}}^{×N}^{ₕ}→ [symbol] ^{N}^{_{ρ}}^{×N}^{_{θ}} represents the Radon transform matrix, when multiplied with the input image f, it outputs the sinogram of the image, s, and ε denotes the system noise. However, patient motion will cause the ideal object f to be degraded to f*=Mf, retaining the following Equation 2

$${s}^{′} = ℛ Mf + ε \qquad ( 2 )$$

where M denotes the motion matrix, and s′ denotes the corrupted sinogram. The motion-corrupted image can be acquired by applying the inverse Radon transform as indicated in Equation 3.

$$f *= {ℛ}^{- 1} {s}^{′} \qquad ( 3 )$$

However, achieving a sharp and fully converged reconstruction may require numerous iterative steps. Reducing the number of iterations leads to a non-ideal solution, f′ shown in Equation 4

$${f}^{′} = f * + δ \qquad ( 4 )$$

where δ denotes a marginal blurring effect resulting from the loss of high-frequency information due to a reduction in the number of iterations. Thus, utilizing fewer iterations during the reconstruction of the corrupted sinogram makes this problem analogous to motion correction as well as a de-blurring problem. The motion corrupted and blurred image, f′, and the corresponding ground truth, f will be utilized for training the GAN.

### Formulation of the Motion Matrix

Let f(x, y) be the original object that is attempting to image, where x and y represent the horizontal and vertical axes, respectively. The model parameterizes the motion with a rotation operator, a and translation operators T_{X}, and T_{Y} in the X-Y direction. Since the image acquisition is being approximated on a slice-by-slice basis, the focus is on rotational motion rather than translational motion along the Z direction.

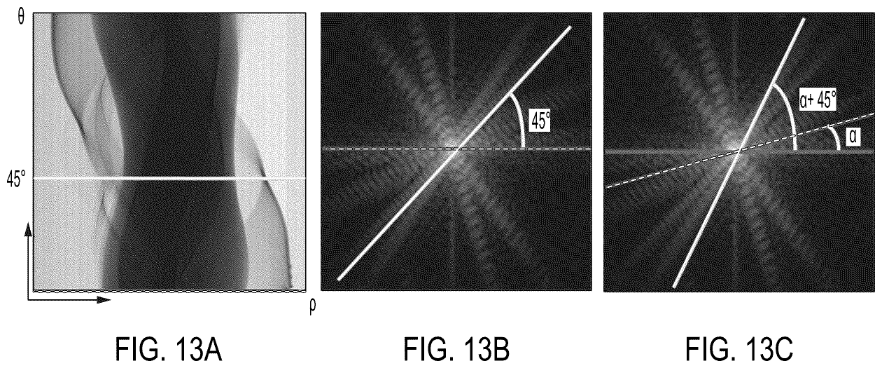

FIGS. 13A-C is an illustration of the Fourier slice theorem. FIG. 13A is a sinogram of a motion free HR-pQCT image. FIG. 1B is a k-space of the corresponding image. FIG. 13C is the k-space of the motion corrupted (rotated by a) image.

Equation 5 shows how the variables, x, and y are altered in the event of motion, M.

$$\begin{matrix}[ \begin{matrix}{x}^{′} \\ {y}^{′} \\ 1\end{matrix} ] & = M [ \begin{matrix}x \\ y \\ 1\end{matrix} ] \\ & = [ \begin{matrix}cos α & - sin α & 0 \\ sin α & cos α & 0 \\ 0 & 0 & 1\end{matrix} ] [ \begin{matrix}{T}_{x} & 0 & 0 \\ 0 & {T}_{y} & 0 \\ 0 & o & 1\end{matrix} ] [ \begin{matrix}x \\ y \\ 1\end{matrix} ] \\ & = [ \begin{matrix}cos α & - sin α & 0 \\ sin α & cos α & 0 \\ 0 & 0 & 1\end{matrix} ] [ \begin{matrix}{T}_{x} x \\ {T}_{y} y \\ 1\end{matrix} ] \\ & = [ \begin{matrix}{T}_{x} x cos α - {T}_{y} y sin α \\ {T}_{x} x sin α + {T}_{y} y cos α \\ 1\end{matrix} ]\end{matrix} \qquad ( 5 )$$

From the Fourier slice theorem, it is imperative that the 1D Fourier transform of the projection signal in parallel beam geometry represents a spoke in that specific angle in the k-space. The formulation of the Fourier slice theorem is shown in Equation 6.

$$S ( θ , ω ) = \int \int ( x , y ) exp ( - j 2 πω ( x cos θ + y sin θ ) ) dx dy = F ( cos θ , sin θ ) \qquad ( 6 )$$

Where, f(x, y) is the object to image, and S(θ, ω) is the 1D Fourier transform of the projection signal at any angle θ. F (cos θ, sin θ) correspond to a particular spoke at projection angle θ in the k-space of the original object f(x, y). This phenomenon is illustrated in FIG. 13A-B where a single line in the sinogram corresponds to a spoke at the corresponding angle in the k-space.

To obtain the motion-corrupted sinogram, the positional variables of the object account for the motion. From Equation 5 the altered position of the object can be calculated caused by rotation and translation, shown in Equation 7.

$${x}^{′} = {T}_{x} x cos α - {T}_{y} y sin α , \qquad ( 7 )$$

$${y}^{′} = {T}_{x} x sin α + {T}_{y} y cos α$$

By differentiating the variables in Equation 7 and substituting the resultant (Equation 8) into the Fourier slice theorem in Equation 6, Equation 9 is obtained, which ultimately simplifies into Equation 10.

$${x}^{′} = {T}_{x} x cos α dx = {T}_{y} y cos α dy \qquad ( 8 )$$

$${S}^{′} ( θ , ω ) = \int \int \frac{f ( {x}^{′} , {y}^{′} )}{{T}_{x} {T}_{y} {cos}^{2} α} exp ( - j 2 πω ( x cos θ + y sin θ ) ) {dx}^{′} {dy}^{′} \qquad ( 9 )$$

$${S}^{′} ( θ , ω ) = \int \int \frac{f ( {x}^{′} , {y}^{′} )}{{T}_{x} {T}_{y} {cos}^{2} α} exp ( - j 2 πω \{ \frac{1}{{T}_{x}} ( {x}^{′} cos α cos θ + {y}^{′} sin α cos θ ) + \frac{1}{{T}_{y}} ( {y}^{′} cos α sin θ - {x}^{′} sin α sin θ ) \} ) {dx}^{′} {dy}^{′} \qquad ( 10 )$$

Given the aforementioned 2D slice approximation, it is assumed that the motion caused by translation is minimal and the same in both axes. Thus, applying T_{X}=T_{Y}=T into Equation 10, Equation 11 is derived.

$${S}^{′} ( θ , ω ) = \int \int \frac{f ( {x}^{′} , {y}^{′} )}{{T}^{2} {cos}^{2} α} \times exp ( - j 2 πω ( \frac{{x}^{′}}{T} cos ( α + θ ) + \frac{{y}^{′}}{T} sin ( α + θ ) ) ) {dx}^{′} {dy}^{′} \approx F ( cos ( θ + α ) , sin ( θ + α ) ) \qquad ( 11 )$$

Thus, in 2D HR-pQCT, the rotation of the peripheral sites leads to the original spokes in k-space being rotated by an angle α, as illustrated in FIG. 13C.

### Simulation of Motion

This section presents a detailed process for simulating motion into a ground truth (non-corrupted) image. The objective is to randomly rotate some of the spokes in k-space to simulate motion. In the context of HR-pQCT, this technique proves to be inefficient due to the necessity of calculating Fourier coefficients, which is computationally demanding.

The current disclosure proposes simulating the motion directly in the sinogram. As each spoke in the k-space corresponds to a line in the sinogram some number of lines in the sinogram can be altered to artificially induce motion. First, an artifact-free, score 1 image, f of size 2304×2304 is resized to manage computational costs. Next, an arbitrary rotation angle is defined, a, to simulate motion. The rotation angles are kept minimal (−π/20 to π/120) based on the assumption that peripheral sites cannot accommodate large rotations. Then, P, the number of projection angles in vector a (projection vector), ranging from 0 to 2π is taken. From the projection angles, assuming parallel beam geometry, the Radon transform can be computed to get the artifact-free sinogram sₐ. Afterward, the projection vector, a is added with an arbitrary rotation angle, α to get a shifted sinogram, Sₐ_{_{shift}}. Finally, L projection signals in sₐ are substituted with the corresponding projection signals from sₐ_{_{shift}} and the corrupted sinogram, s′ is received. This procedure is analogous to rotating certain spokes in k-space by arbitrary angles, as depicted in FIG. 13A-C. Finally, Simultaneous Iterative Reconstruction Technique (“SIRT”) is utilized to reconstruct the motion-corrupted image, f′ from the corrupted sinogram, s′. The detailed explanation of the motion simulation steps for a single image is outlined in Process 1.

Process 1

| | |
|---|---|
| 1: | Input: Artifact-free image, f |
| 2: | Output: Corrupted image, f′ |
| 3: | Resize the input, finto (512, 512) |
| 4: | $Take arbitrary N rotation angles , {\{ α \}}_{i = 1}^{N}$ |
| 5: | Construct a projection vector containing P number of linearly |
| | spaced projection angles, a ∈ [symbol] ^{P×1}, αᵢ∈[0,2π] |
| 6: | sₐ = [symbol] (a), [symbol] ( ) = Radon transform |
| 7: | Choose a random rotation angle, |
| | $j = 1 , … , N from {\{ α \}}_{i = 1}^{N}$ |
| 8: | a_{shift} = a + αⱼ |
| 9: | sₐ_{_{shift}} = [symbol] (a_{shift}) |
| 10: | Select a random integer, m from m = 1, 2, 3, . . . , (512-L), |
| | L = the number corrupted lines, |
| 11: | sₐ (m : m + L) ← sₐ_{_{shift}} (m:m + L) |
| 12: | s′ ← sₐ |
| | {SIRT Algorithm} |
| | {BEGIN} |
| 13: | Initiate v⁽⁰⁾ = 0 |
| 14: | Set number of intertions, iter |
| 15: | P ← s′ |
| 16: | for k = 1, . . . , iterdo |
| 17: | v^{(k)} ← v^{(k-1)} + CW^{T} R(p − Wv^{(k-1)}), where W is the forward |
| | projection matrix, C, and R are the weights associated with the |
| | projection. |
| 18: | end for |
| 19: | f′ ← v^{(iter)} |
| | {END} |
| 20: | Extract the ROI, and resize, f′ into (256,256) |
| 21: | return f′ |

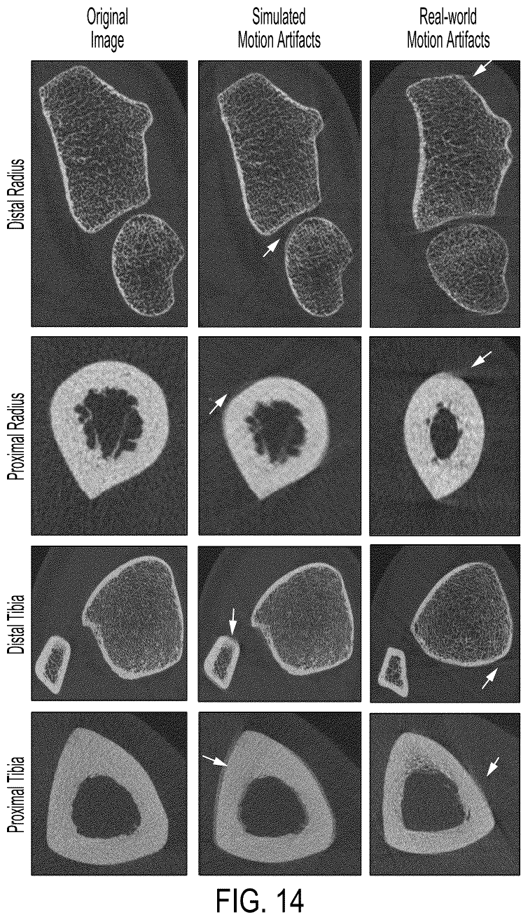

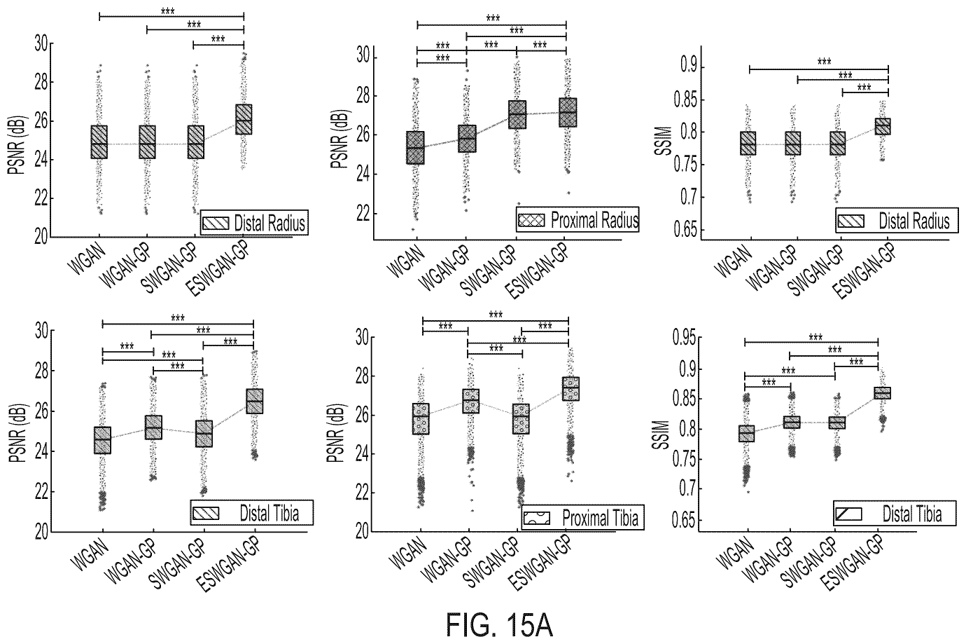

The rotation angle α and the number of lines to be substituted, L, are significant parameters to adjust in order to simulate varying levels of motion. The outcome of the proposed simulation method is illustrated in FIG. 14, where four peripheral scan sites are exposed to various degrees of motion simulation and are compared with real-world motion-corrupted data from the scanner.

FIG. 14 represents motion artifacts that are simulated utilizing the proposed sinogram-based motion simulation framework. The first two columns display the motion score 1 (motion-free image) alongside the corresponding simulated motion artifacts in the same participant, while the third column represents a real-world motion scenario in a different participant. The arrows denote the particular artifacts that this study aims to address. The Region of Interest (“ROI”) is cropped to display only the bone area in the figure.

### Proposed Model for Motion Correction

Following the motion simulation scheme, 483 pairs of simulated motion-corrupted and ground-truth data were obtained from four different peripheral sites. Each pair includes 168 ground-truth images and 168 simulated motion-corrupted images. The Region of Interest (“ROI”) was extracted from these image pairs, resized to 256×256, and subsequently processed by the proposed ESWGAN-GP model, which maps the motion-corrupted images to their corresponding ground truth, effectively correcting the motion artifacts in the process. During the training, the predicted results are compared with the ground-truth to calculate the loss, which is subsequently back-propagated to update the network parameters. Specifically, the motion-corrupted image, f′ undergoes a deep learning network parameterized with G_{θ}. to predict a motion-free image f\hat{ } as shown in equation 12.

$$\hat{f} = {G}_{θ} ( {f}^{′} ) \qquad ( 12 )$$

An objective function or loss is minimized to obtain the optimal θ\hat{ }, ensuring that G_{θ} predicts a motion-free image, f\hat{ } that closely approximates the corresponding ground-truth, f.

$$\hat{θ} = arg {min}_{θ} {\sum }_{i} ℒ ( {\hat{f}}_{i} ( θ ) , {f}_{i} ) \qquad ( 13 )$$

The backbone of the proposed model is the Generative Adversarial Network (“GAN”) which includes a generator network, G, and a discriminator network, D. The generator network G, maps the input F′ to F i.e., G:F′→F(G), where f′∈F′, and f∈F. The generator's task is to predict a motion-compensated image, f\hat{ }, from the input image f\hat{ }, while the discriminator, D, attempts to distinguish between the generated motion-compensated image, f\hat{ } and the ground truth image, f. Training continues until the generator is able to fool the discriminator, making it unable to differentiate between the generated motion-compensated image and the ground truth motion-free image. The following optimization problem is addressed during the training process.

$${min}_{G} {max}_{D} {ℒ}_{GAN} ( G , D ) \qquad ( 14 )$$

The subsequent sections are organized as follows, Section 1 provides a detailed explanation of the adversarial loss, while Section 2 offers a concise overview of the content loss.
1) Adversarial Loss: Unlike the negative log-likelihood used in traditional GANs, the present disclosure employs the earth-mover distance or Wasserstein GAN (“WGAN”) to ensure differentiability with respect to the input, thereby mitigating the training difficulties associated with log-likelihood loss. In particular, the discriminator's gradient with respect to the input is optimized more effectively in WGANs than in standard GANs, leading to improved performance of the generator. Further improvements can be achieved by integrating Gradient Penalty into WGAN (WGAN-GP), which modifies the optimization problem to the following form.

$${min}_{G} {max}_{D} {ℒ}_{WGAN - GP} ( G , D ) = {𝔼}_{{f}^{′}} [ D ( G ( {f}^{′} ) ) ] - {𝔼}_{{f}^{′}} [ D ( {f}^{′} ) ] + {λ𝔼}_{\overline{f}} ⌈ {( {‖ {∇}_{\overline{f}} D ( \tilde{f} ) ‖}_{2} - 1 )}^{2} ⌉ \qquad ( 15 )$$

Here, equation (15) represents the expectation operator. The first two terms correspond to the discriminator's criteria, where both the generated image and the corresponding ground truth image are input into the discriminator, and the Wasserstein distance is measured. Conversely, the third term represents the gradient penalty, which enforces 1-Lipschtiz constraint i.e., the gradients of the discriminator output with respect to input are normalized and constrained to remain below 1; any deviation beyond this threshold incurs a penalty.
2) Content Loss: In addition to the generator loss, a VGG-based perceptual 11-loss is employed, where both the ground truth image and the generated image are processed through a VGG network, which is pre-trained on a natural image dataset, Imagenet. The content loss function is shown in equation 16.

$${ℒ}_{content} ( G ) = η \times - 𝔼 ( D ( G ( {f}^{′} ) ) ) + {𝔼}_{( {f}^{′} , f )} [ {‖ VGG ( G ( {f}^{′} ) ) - VGG ( g ) ‖}_{1} ] \qquad ( 16 )$$

In addition to the adversarial loss, the final optimization objective for this problem is formulated as follows:

$${min}_{G} {max}_{D} {ℒ}_{WGAN - GP} ( G , D ) = {𝔼}_{{f}^{′}} [ D ( G ( {f}^{′} ) ) ] - {𝔼}_{{f}^{′}} [ D ( {f}^{′} ) ] + {λ𝔼}_{\overline{f}} [ {( {‖ {∇}_{\overline{f}} D ( \tilde{f} ) ‖}_{2} - 1 )}^{2} ] + {ℒ}_{content} \qquad ( 17 )$$

### The Proposed Network Architecture

The backbone of the generator is a U-Net architecture, consisting of an encoder block that employs multiple convolutional layers to transform the input into feature space, and a decoder block that reconstructs the images from the lower-level feature information. Skip connections are implemented between various layers of the encoder and decoder to prevent the vanishing gradient problem and maintain consistency with the original input. In addition to the skip connections originally proposed in the U-Net architecture, an edge enhancer block is introduced that transfers edge information directly from the input to the final output. This architecture effectively captures both low-level details through skip connections and global structure through down sampling and up sampling operations, while the additional edge enhancement block helps preserve cortical and trabecular edge features. Moreover, a self-attention module is utilized within both the generator and discriminator to effectively capture long-range dependencies within an image. Incorporating attention blocks enables the evaluation of an image based on its global context, as opposed to the localized focus typically employed by conventional CNNs. The proposed network architecture is shown in FIGS. 3A-B. The subsequent sections provide detailed descriptions of the edge enhancer block and the attention network. The architecture flowchart can also be seen in FIG. 4.
1) Edge-enhancer Block: A Sobel-kernel-based Convolutional Neural Network (SCNN) module is incorporated along-side the skip connection layer in the uppermost layer of the U-Net-based generator for edge enhancement. In SCNN, the input image undergoes Sobel kernel filtering to generate an edge-detected image. This edge-detected image is then subjected to a sequence of three consecutive convolutional layers followed by rectified Linear Units (ReLU) before being combined through matrix addition with the final output of the decoder (SCNN in FIG. 3) 604. To derive the edge image from the input, the Sobel kernel employs two distinct filters oriented along the X and Y directions. Convolution with these filters is followed by calculating the magnitude squared image, which results in the final edge-detected image. The rationale for incorporating an edge enhancement module into the deep learning network is grounded in the high-resolution nature of HR-pQCT, which necessitates precise edge reconstruction to ensure measurement of cortical porosity and thickness as well as trabeculae thickness.
2) Self-attention Block: The proposed approach incorporates self-attention blocks into specific layers of the generator and discriminator (white blocks shown in FIGS. 3A-B) to effectively capture long-range dependencies within an image. The attention block, in the generator, is connected directly to the feature maps produced by lower convolutional layers rather than to a flattened output from a fully connected layer, or the upper layers in the generator. This design choice is intentional: by working with 2D feature maps, the attention block can selectively focus on certain regions or patterns within the spatial structure of the image. This setup enables the attention mechanism to emphasize important parts of the image while maintaining the original spatial relationships between pixels, which would be lost if the data were flattened or data were still in the low-level feature state, e.g., in the upper portion of the generator. As a result, the attention block can better highlight relevant features and patterns within the 2D layout of the image.

FIGS. 3A-B is an illustration of the proposed ESWGAN-GP network, which includes a generator and a discriminator. The motion-corrupted image, f′, is fed into the generator, which encodes it into a feature space with dimensions 32×32×512. This encoded feature is then passed through the decoder, which reconstructs it back to the original input image dimensions (256). In the final step, the SCNN's input and output are combined element-wise with the decoded feature to generate the predicted motion-compensated image, G_{θ}(f′). In SCNN, the input image's edges are extracted using a Sobel-kernel convolution and processed through a series of convolutional and activation layers to produce feature maps with the same spatial dimensions as the input image. The predicted image, G_{θ}(f′) is passed through the discriminator alongside its motion-free counterpart, f, and a Wasserstein distance is utilized to minimize the distance. The dotted lines indicate connections originating from the preceding layer.

### EXPERIMENTAL DETAILS AND RESULTS

In this section, the dataset employed in this study is described, followed by a detailed explanation of the experimental setup, including both the motion simulation parameters and the ESWGAN-GP parameters. Subsequently, an ablation study is conducted to assess the contribution of each component of the ESWGAN-GP network. Finally, the proposed motion correction scheme is validated using both a simulated motion dataset (source) and a real-world motion-corrupted dataset (target). For quantitative evaluation, PSNR, SSIM, and VIF metrics are used on the simulated dataset where ground truth data is available. These metrics were also used for assessing the reconstruction performance on the target domain. The target domain input, which has reduced iteration during reconstruction with inherent motion artifacts, is compared with their original raw image through these metrics to measure data fidelity—specifically, the retention of micro-structural information despite motion correction.

### Dataset

Between 2018 and 2022, a cohort of 559 volunteers who underwent HR-pQCT scanning through the Musculoskeletal Function, Imaging, and Tissue Resource Core (FIT Core) at the Indiana Center for Musculoskeletal Health's Clinical Research Center (Indianapolis, Indiana) met the criteria for initial inclusion in this study, with selection being random and including motion grades of all types, 1 to 5. The FIT Core received Institutional Review Board (IRB) approval from Indiana University, and each participant provided written informed consent prior to imaging. HR-pQCT scans (XtremeCT II, Scanco Medical, Bruttisellen, Switzerland) were acquired on the non-dominant arm at 4% and 30% the bone length from the radius reference line, and at 7.3% and 30% from the tibia reference line, as previously described. Participants were positioned supine on a movable treatment plinth, with the limb of interest stabilized using padded carbon fiber casts provided by the manufacturer. Participants were instructed to remain motionless during scanning. Scanning parameters included 68 kVp and 1.47 mA, with 168 slices (covering 10.2 mm of bone) acquired at a voxel size of 60.7 μm. Scanner stability was maintained by routinely scanning phantoms with density and volume inserts as per the manufacturer's guidelines. Motion was assessed by a trained operator using a visual grading score (“VGS”), ranging from 1 (no motion artifacts) to 5 (severe streaking, cortical disruptions, and trabecular blurring).

TABLE I
provides a comprehensive summary of participants
and the corresponding images used in the study.

| | Train-Test | | |
|---|---|---|---|
| Bone Type | Split | Participants | Images |
| Distal (4%) | Train (Source) | 90 | 15,120 |
| Radius | Test (Source) | 13 | 2,184 |
| | Test (Target) | 41 | 6,888 |
| Proximal (30%) | Train (Source) | 100 | 16,800 |
| Radius | Test (Source) | 30 | 5,040 |
| | Test (Target) | 13 | 2,184 |
| Distal (7.3%) | Train (Source) | 90 | 15,120 |
| Tibia | Test (Source) | 36 | 6,048 |
| | Test (Target) | 14 | 2,352 |
| Proximal (30%) | Train (Source) | 90 | 15,120 |
| Tibia | Test (Source) | 34 | 5,712 |
| | Test (Target) | 8 | 1,344 |
| | Total | 559 | 93,912 |

The table shows the summary of the dataset utilized in this study. The source data includes VGS 1 images used for simulating motion artifacts, thereby containing ground truth information. Conversely, the target data comprises real-world motion-corrupted images of VGS-2, 3, 4, and 5. Images from the same participants are strictly segregated between the training and testing sets, avoiding any patient overlap in the train-test split across all experiments.

In some examples, patient data is used to build/train models may be obtained from existing scans in a patient database or other preexisting scan (e.g., as DICOM images). In some cases, data may be preprocessed, such as interpolated to a common number of frames. Continuing the example, each subject's data may be under sampled or oversampled to simulate the acquisition with different lower or higher temporal resolutions, as discussed above. Model parameters may be tuned and fitted, e.g., based on the data and the original data, using deep learning toolkits, such as, for example high performance GPU workstations and software packages.

### EXPERIMENTAL DETAILS

1) Motion Simulation Parameters: The sinogram-based motion simulation proposed in Section II-B involves two variables: a, representing the rotation angle of the imaging object, and L, denoting the number of lines altered in the sinogram due to motion. The rotation angles α are selected from a range of values, specifically from −π/20 to π/120. Out of the 1800 total projections, 200 consecutive projections were altered to introduce motion artifacts. The motion simulation was implemented using MATLAB 2023a with the help of ASTRA Toolbox.
2) Neural Network Parameters: In the proposed ESWGAN-GP, the penalty term in the discriminator is governed by the parameter λ, which manages the balance between the Wasserstein loss and the gradient penalty. For all experiments, λ is set to 0.2. Additionally, the ADAM optimizer is employed, with decay rates set to β1=0.5 and β2=0.999. The learning rate is fixed at 8×10−5. The batch size for all experiments is set to 1. The trade-off between the generator loss and VGG-based perceptual loss is controlled by the parameter, η=1×10−3. All the codes for the neural network were implemented using the PyTorch framework, and the simulations were conducted on an NVIDIA RTX A5500 GPU with 24 GB of memory.

### Results

First, the selection of the proposed network, ESWGAN-GP is evaluated by comparing it to its preceding models: 1) WGAN, 2) WGAN-GP, and 3) SWGAN-GP. Consistency in the content loss has been maintained across all networks, incorporating both the generator loss and the VGG-based perceptual loss. For the ablation study, all four peripheral sites are utilized, and tests are conducted on simulated, and target data. The mean PSNR, SSIM, and VIF values, along with their corresponding standard deviations are calculated. Secondly, the performance of the proposed ESWGAN-GP is compared with GAN-CIRCLE, a previously applied method for CT super-resolution, after making slight modifications to adapt it to the proposed dataset for performance evaluation. Due to the unavailability of code for the only existing motion correction abstract it was opted to implement GAN-CIRCLE as the most suitable alternative for comparison.

TABLE IIA
Comparison on PSNR, SSIM, and VIF values across different sites for four different models

| | Distal | | | Proximal Radius | | |
|---|---|---|---|---|---|---|
| | PSNR | SSIM | VIF | PSNR | SSIM | VIF |
| WGAN (S) | 25.15 ± 1.24 | 0.78 ± 0.02 | 0.58 ± 0.04 | 25.38 ± 1.16 | 0.71 ± 0.03 | 0.59 ± 0.04 |
| WGAN (T) | 28.62 ± 1.07 | 0.84 ± 0.01 | 0.64 ± 0.03 | 29.00 ± 0.84 | 0.81 ± 0.04 | 0.76 ± 0.03 |
| WGAN-GP | 25.15 ± 1.24 | 0.78 ± 0.02 | 0.58 ± 0.04 | 25.28 ± 0.97 | 0.78 ± 0.04 | 0.75 ± 0.04 |
| (S) | | | | | | |
| WGAN-GP | 29.26 ± 1.01 | 0.81 ± 0.01 | 0.64 ± 0.03 | 28.60 ± 0.74 | 0.79 ± 0.03 | 0.72 ± 0.03 |
| (T) | | | | | | |
| SWGAN- | 25.15 ± 1.24 | 0.78 ± 0.03 | 0.58 ± 0.05 | 25.00 ± 1.00 | 0.75 ± 0.02 | 0.70 ± 0.03 |
| GP (S) | | | | | | |
| SWGAN- | 28.62 ± 1.07 | 0.88 ± 0.01 | 0.77 ± 0.04 | 29.73 ± 0.64 | 0.82 ± 0.03 | 0.82 ± 0.02 |
| GP (T) | | | | | | |
| ESWGAN- | 25.15 ± 1.24 | 0.78 ± 0.02 | 0.58 ± 0.04 | 25.38 ± 1.16 | 0.71 ± 0.03 | 0.59 ± 0.04 |
| GP (S) | | | | | | |
| ESWGAN- | 29.83 ± 1.00 | 0.87 ± 0.01 | 0.77 ± 0.04 | 29.85 ± 1.05 | 0.90 ± 0.01 | 0.87 ± 0.02 |
| GP (T) | | | | | | |
| GAN- | 24.18 ± 1.32 | 0.73 ± 0.04 | 0.55 ± 0.04 | 24.27 ± 1.37 | 0.73 ± 0.03 | 0.59 ± 0.05 |
| CIRCLE | | | | | | |
| (S) | | | | | | |
| GAN- | 27.45 ± 1.09 | 0.81 ± 0.02 | 0.61 ± 0.04 | 28.49 ± 0.72 | 0.82 ± 0.02 | 0.59 ± 0.04 |
| CIRCLE | | | | | | |
| (T) | | | | | | |

TABLE IIB
Comparison on PSNR, SSIM, and VIF values across different sites for four different models

| | Distal Tibia | | | Proximal Tibia | | |
|---|---|---|---|---|---|---|
| | PSNR | SSIM | VIF | PSNR | SSIM | VIF |
| WGAN | 24.35 ± 0.98 | 0.79 ± 0.02 | 0.59 ± 0.03 | 25.68 ± 1.10 | 0.74 ± 0.03 | 0.53 ± 0.04 |
| (S) | | | | | | |
| WGAN | 27.15 ± 0.84 | 0.86 ± 0.02 | 0.68 ± 0.03 | 28.43 ± 0.85 | 0.81 ± 0.04 | 0.59 ± 0.03 |
| (T) | | | | | | |
| WGAN-GP | 25.15 ± 0.85 | 0.81 ± 0.02 | 0.65 ± 0.03 | 25.58 ± 0.87 | 0.77 ± 0.07 | 0.71 ± 0.03 |
| (S) | | | | | | |
| WGAN-GP | 28.35 ± 0.78 | 0.85 ± 0.02 | 0.73 ± 0.03 | 27.99 ± 0.59 | 0.80 ± 0.04 | 0.71 ± 0.03 |
| (T) | | | | | | |
| SWGAN- | 27.40 ± 1.10 | 0.87 ± 0.02 | 0.70 ± 0.03 | 25.67 ± 1.10 | 0.74 ± 0.03 | 0.52 ± 0.03 |
| GP (S) | | | | | | |
| SWGAN- | 26.70 ± 0.88 | 0.88 ± 0.02 | 0.74 ± 0.03 | 28.15 ± 0.98 | 0.84 ± 0.03 | 0.79 ± 0.03 |
| GP (T) | | | | | | |
| ESWGAN- | 24.35 ± 0.98 | 0.79 ± 0.02 | 0.59 ± 0.03 | 25.68 ± 1.10 | 0.74 ± 0.03 | 0.53 ± 0.04 |
| GP (S) | | | | | | |
| ESWGAN- | 29.17 ± 0.70 | 0.90 ± 0.02 | 0.80 ± 0.02 | 28.54 ± 1.03 | 0.87 ± 0.03 | 0.81 ± 0.03 |
| GP (T) | | | | | | |
| GAN- | 28.35 ± 0.97 | 0.73 ± 0.03 | 0.58 ± 0.03 | 25.90 ± 1.36 | 0.77 ± 0.01 | 0.53 ± 0.04 |
| CIRCLE | | | | | | |
| (S) | | | | | | |
| GAN- | 26.34 ± 0.84 | 0.80 ± 0.02 | 0.68 ± 0.03 | 27.40 ± 0.53 | 0.78 ± 0.01 | 0.59 ± 0.03 |
| CIRCLE | | | | | | |
| (T) | | | | | | |

TABLE IIIA
Statistical analysis using ANOVA and Tukey-Kramer for PSNR, SSIM, and
VIF scores across different regions in the source dataset. A check
indicates statistical significance at a p-value < or equal to 0.01.

| | Distal Radius | | | Proximal Radius | | |
|---|---|---|---|---|---|---|
| | PSNR | SSIM | VIF | PSNR | SSIM | VIF |
| WGAN vs WGAN-GP | X | X | X | ✓ | ✓ | ✓ |
| WGAN vs SWGAN-GP | X | X | X | ✓ | ✓ | ✓ |
| WGAN vs ESWGAN-GP* | ✓ | ✓ | ✓ | ✓ | ✓ | ✓ |
| WGAN-GP vs SWGAN-GP | X | ✓ | ✓ | ✓ | ✓ | ✓ |
| WGAN-GP vs ESWGAN- | ✓ | ✓ | ✓ | ✓ | ✓ | ✓ |
| GP* | | | | | | |
| SWGAN-GP vs ESWGAN- | ✓ | ✓ | ✓ | ✓ | ✓ | ✓ |
| GP* | | | | | | |
| GAN-CIRCLE vs | ✓ | ✓ | ✓ | ✓ | ✓ | ✓ |
| ESWGAN-GP* | | | | | | |

TABLE IIIB
Statistical analysis using ANOVA and Tukey-Kramer for PSNR, SSIM, and
VIF scores across different regions in the source dataset. A check
indicates statistical significance at a p-value < or equal to 0.01.

| | Distal Tibia | | | Proximal Tibia | | |
|---|---|---|---|---|---|---|
| | PSNR | SSIM | VIF | PSNR | SSIM | VIF |
| WGAN vs WGAN-GP | ✓ | ✓ | ✓ | ✓ | ✓ | ✓ |
| WGAN vs SWGAN-GP | ✓ | ✓ | ✓ | X | X | X |
| WGAN vs ESWGAN-GP* | ✓ | ✓ | ✓ | ✓ | ✓ | ✓ |
| WGAN-GP vs SWGAN-GP | ✓ | X | ✓ | ✓ | ✓ | ✓ |
| WGAN-GP vs ESWGAN- | ✓ | ✓ | ✓ | ✓ | ✓ | ✓ |
| GP* | | | | | | |
| SWGAN-GP vs ESWGAN- | ✓ | ✓ | ✓ | ✓ | ✓ | ✓ |
| GP* | | | | | | |
| GAN-CIRCLE vs | ✓ | ✓ | ✓ | ✓ | ✓ | ✓ |
| ESWGAN-GP* | | | | | | |

1) Ablation Study: From Table II, the superior performance of ESWGAN-GP is seen across the four anatomical sites in the source(S), and target (T) dataset. Specifically, the ESWGAN-GP(S) achieves the highest PSNR and SSIM values consistently across all regions, with VIF values also reflecting improved fidelity in the source domain. A similar trend can also be observed in the target domain, i.e., ESWGAN-GP (T) outperforming all the other models. Statistical analysis by ANOVA and Tukey-Kramer post hoc analysis indicates that ESWGAN-GP results in higher image quality than the other three models, as shown in Table III. Given ESWGAN-GP's superior performance across all four peripheral sites, as shown in FIG. 4, this model was selected for its enhanced capabilities. However, the preceding models also demonstrate potential utility as effective methods for motion correction.

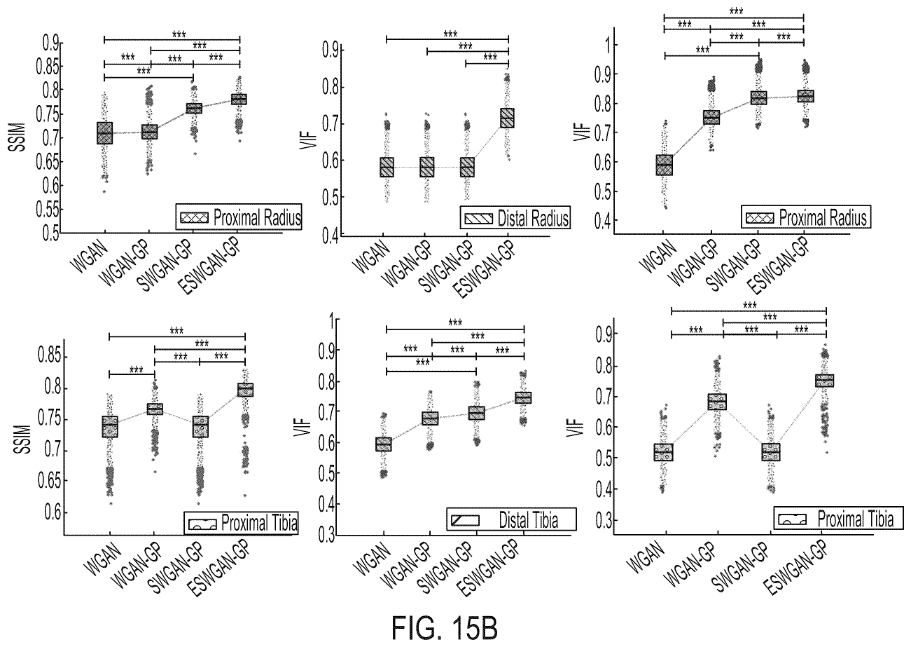

FIGS. 15A-B compares the PSNR, SSIM, and VIF values of four models (WGAN, WGAN-GP, SWGAN-GP, and ESWGAN-GP) across four anatomical sites (Distal Radius, Proximal Radius, Distal Tibia, and Proximal Tibia) in the source dataset. Each plot represents the performance of the four models at a specific site. Red stars indicate outliers in the data. The symbol ‘***’ denotes a p-value of ≤0.01, as determined by a paired t-test under the assumption of unequal variance. Pairs that are not connected indicate a lack of statistically significant difference.

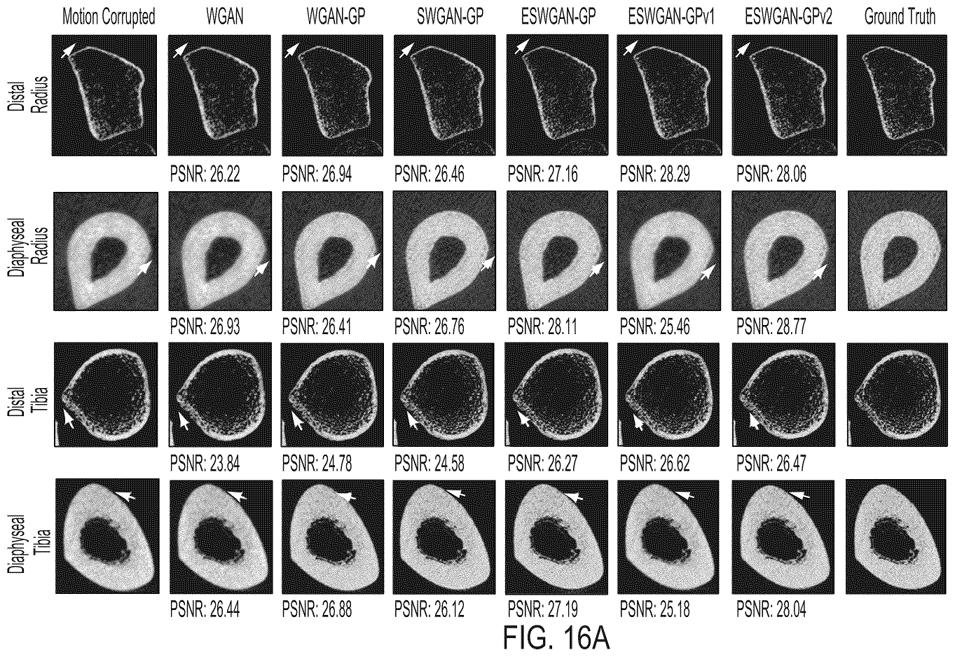

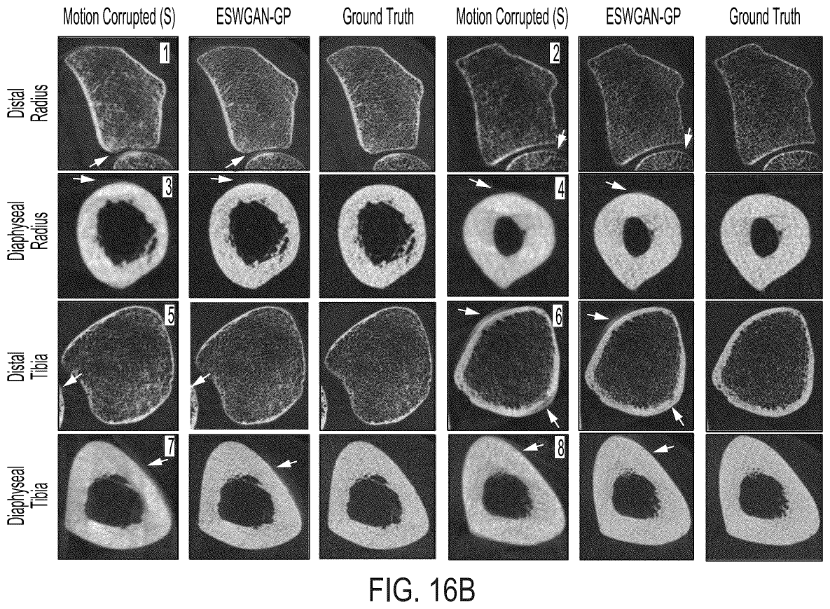

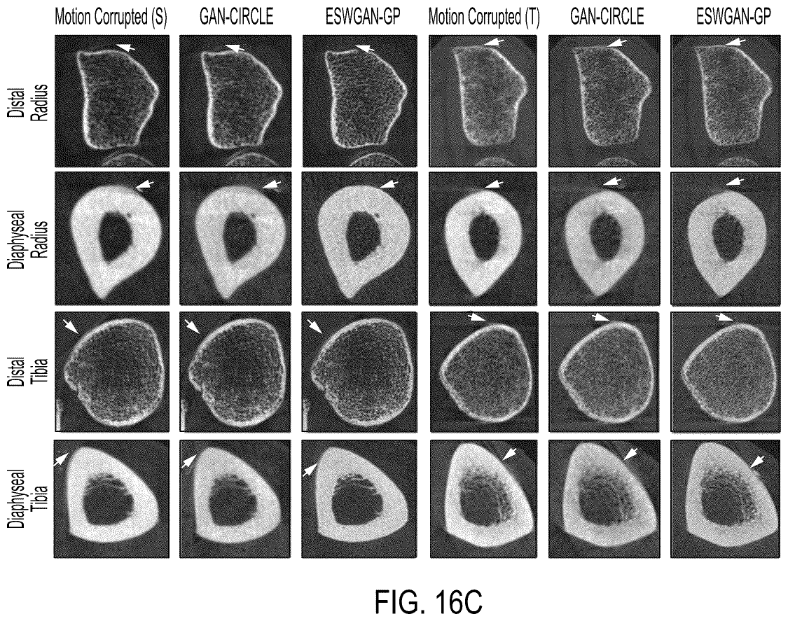

FIG. 16A presents a qualitative analysis from the ablation study, highlighting the progression of motion correction achieved through the four evaluated models. The figure reveals a consistent trend: the incorporation of a gradient penalty enhances network stability, leading to improved outcomes compared to WGANs at all four sites. Notably, the inclusion of the self-attention block in SWGAN-GP results in better cortical bone reconstruction, particularly in the proximal radius and distal tibia. Furthermore, the final enhancement comes from the edge enhancer block (SCNN), which significantly improves the reconstruction of fine microstructural details, most notably in the distal radius and distal tibia.
2) Comparison with GAN-CIRCLE: As seen in Table II, ESWGAN-GP outperforms GAN-CIRCLE by approximately 2.5 dB in PSNR on the source dataset and demonstrates an average improvement of 1.8 dB on the target dataset. Statistical analysis in Table III further claims that the mean metric values of the ESWGAN-GP outperform GAN-CIRCLE. This can be attributed to the design choices in the two models. GAN-CIRCLE relies on cycle-consistency, which forces the generated data to closely resemble the source data, thereby preserving consistency with the input. However, resolving motion artifacts does not necessitate maintaining such strict input consistency.

FIG. 16B illustrates a second set of comparative results. Here, GAN-CIRCLE is compared to ESWGAN-GP on both source images (e.g., simulated images) and target images (e.g., real-world motion corrupted data). As illustrated, when both models were trained on source data and applied to real-world motion corrupted data (“Motion Corrupted (T)”), ESWGAN-GP outperforms GAN-CIRCLE and provides results comparable to its results on source data.

### Discussion

Owing to its capacity to deliver detailed evaluations of cortical and trabecular bone microstructure and mineralization, HR-pQCT is increasingly favored for fracture risk prediction in osteoporosis. It serves as a valuable complement to a BMD measurements obtained from DXA, while also enabling the detection of bone mineral alterations associated with conditions such as chronic kidney disease (“CKD”), thus providing a more comprehensive assessment of bone fragility. However, its performance can be severely hindered by motion artifacts, which remain a significant challenge. Repeated scans increase the time burden on both staff and patients and frequently fail to mitigate artifacts leading to compromised data quality.

Although advancements in motion grading have been made, the lack of robust motion correction methods continues to obstruct accurate interpretation of key bone parameters. Motion-compromised images fail to represent true bone structure, thereby limiting the potential of HR-pQCT in clinical and research settings. Addressing this limitation is essential to enhance the reliability of HR-pQCT data and fully realize its promise as a non-invasive tool for bone health assessment.

As described herein, a motion simulation and correction model for HR-pQCT bone imaging is provided. The motion simulation model was employed to generate pairs of motion-corrupted and motion-free (ground truth) images, which were then used for training within a supervised learning framework. Experimental results demonstrate that the motion correction framework effectively eliminates artifacts in cortical bone images and improves the visualization of trabecular bone architecture.

As described herein, a sinogram-based approach may be used to simulate motion artifacts in HR-pQCT, establishing a standardized degradation model. By employing an Edge-enhanced Self-attention Wasserstein Generative Adversarial Network with Gradient Penalty (ESWGAN-GP), the techniques effectively address motion artifacts in both simulated and real-world data. The integration of edge-enhancing skip connections, self-attention mechanisms, and a VGG-based perceptual loss contributes to the accurate reconstruction of fine microstructural features. Qualitative evaluations confirm the validity of the motion-corrupted images generated by the simulation, and quantitative metrics demonstrate the potential of the ESWGAN-GP in partially correcting motion artifacts. The technology described herein employs deep learning in motion correction for HR-pQCT, enabling a reduction in the need for patient rescans, thereby enhancing the clinical utility and adoption of this imaging modality.

FIG. 16C shows the proposed ESWGAN-GP is compared with GAN-CIRCLE in both the simulated unseen test data and the target unseen test data. The motion artifacts, highlighted by arrows, visually illustrate how these artifacts are resolved in the two networks. The corresponding difference images are not included in this figure.

Two primary factors limit the realism of the simulation method. First, the image resolution has been significantly reduced from 2304×2304 pixels to 256×256 pixels due to computational limitations. Secondly, the proposed motion simulation process prioritizes in-plane rotational motion, in contrast to the model developed by others that incorporates both in-plane (i.e., x-y plane) and longitudinal (i.e., z-axis) translational components. This restricts the practical applicability of the proposed method. Nonetheless, in-plane translation can be incorporated by quantifying sinogram mismatches between parallelized projections, as described in other studies, while z-axis translation can be simulated by substituting projection lines from adjacent slices, effectively modeling displacement along the acquisition axis. Since these translational components can be integrated within the existing framework, future efforts will aim to generate more realistic and diverse datasets, thereby improving the utility of data-driven motion correction strategies. Importantly, the proposed ESWGAN-GP architecture is agnostic to the specific type of motion simulation, enabling training on datasets derived from alternative modeling techniques. Future work will leverage this flexibility by combining simulation methods that include both the current approach and that of others to develop a more generalizable and robust deep learning-based solution for motion correction.

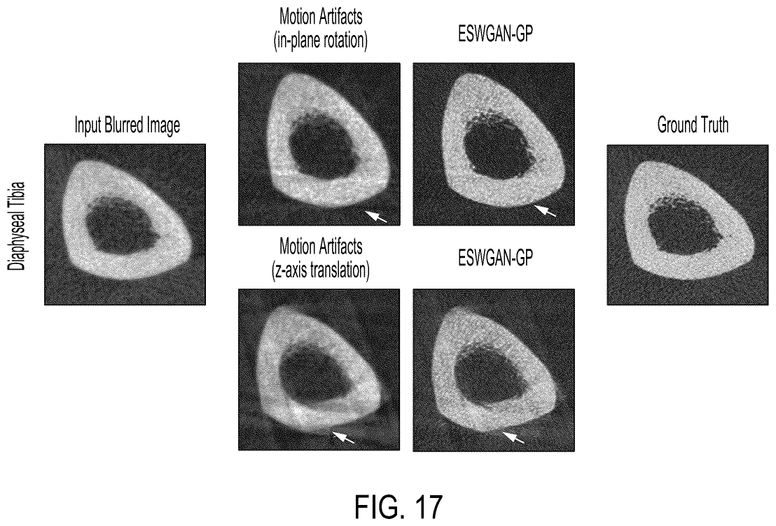

Nonetheless, the effectiveness of ESWGAN-GP in correcting motion artifacts resulting from translational motion along the z-axis was evaluated. As illustrated in FIG. 17, the model's performance was assessed using artifacts generated through manual in-vivo simulation of both in-plane rotation and z-axis translation. The results demonstrate that ESWGAN-GP effectively restores cortical boundaries disrupted by in-plane rotation and exhibits a limited ability to correct motion artifacts resulting from z-axis translation. However, under conditions of substantial translational motion, as observed in the second row of FIG. 17 slight bending of the cortical boundary remains evident when compared to the ground truth. These findings underscore the need for a more robust and comprehensive simulation framework to enable effective correction of motion artifacts in HR-pQCT imaging.

FIG. 17 shows a qualitative evaluation of the robustness of ESWGAN-GP. A volunteer was instructed to simulate in-plane rotational motion (top row) and z-axis translational motion (bottom row) during image acquisition. For ground truth reference, the same volunteer was scanned while being held securely to minimize motion. Corresponding difference images are not displayed in this figure.

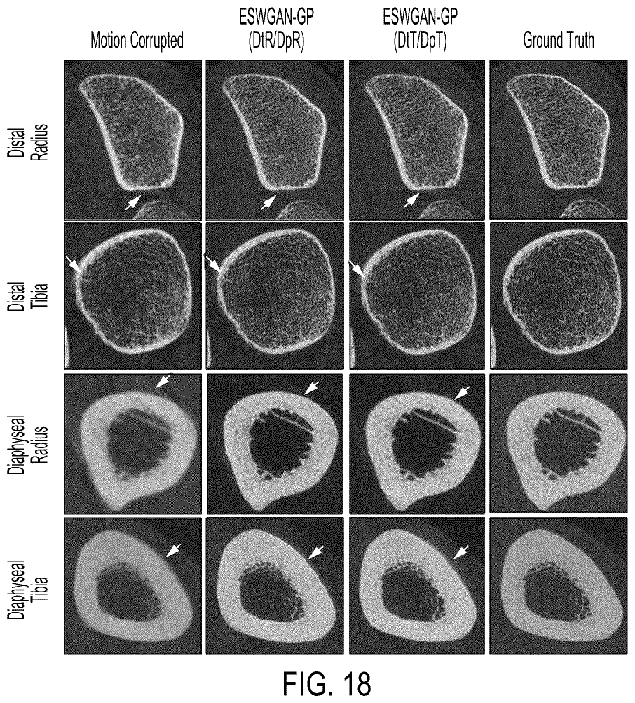

FIG. 18 shows a qualitative evaluation of the model generalizability in the unseen test data from the source domain is performed. ESWGAN-GP (“DtR/DpR”) indicates training on Distal or Dia-physeal Radius data, and ESWGAN-GP (“DtT/DpT”) on Distal or Diaphyseal Tibia data, with the first two rows representing distal sites and the last two diaphyseal sites. The white arrow highlights the artifacts targeted in this study; difference images are not shown.

In this study, the ESWGAN-GP model, along with its variants used in the ablation study, was trained on data from four distinct anatomical sites and evaluated on the corresponding sites. Given the morphological similarities between the Distal Radius and Tibia, such as thin cortical bone and dense trabecular architecture, as well as between the Diaphyseal Radius and Tibia, where the cortical bone tends to be thicker with reduced trabecular density, the model's generalizability was assessed through cross-site evaluations. Specifically, the ESWGAN-GP model trained on the Radius was tested on the Tibia, and vice versa. The outcomes of this cross-site evaluation are presented in Table IV.

TABLE IV
Evaluation of model generalizability using PSNR, SSIM, and VIF metrics across cross-
site training and testing on radius and tibia. The rows above the midline represent
distal site experiments; those below represent diaphyseal site experiments.

| | Distal/Diaphyseal Radius | | | Distal/Diaphyseal Tibia | | |
|---|---|---|---|---|---|---|
| | PSNR | SSIM | VIF | PSNR | SSIM | VIF |
| ESWGAN-GP | 26.37 ± 1.13 | 0.81 ± 0.02 | 0.71 ± 0.04 | 24.98 ± 0.93 | 0.81 ± 0.02 | 0.72 ± 0.03 |
| (DtR) (S) | | | | | | |
| ESWGAN-GP | 28.60 ± 1.28 | 0.83 ± 0.02 | 0.69 ± 0.04 | 26.45 ± 0.88 | 0.86 ± 0.02 | 0.75 ± 0.03 |
| (DtT) (S) | | | | | | |
| ESWGAN-GP | 29.23 ± 1.00 | 0.87 ± 0.01 | 0.77 ± 0.03 | 27.30 ± 0.81 | 0.86 ± 0.01 | 0.78 ± 0.03 |
| (DtR) (T) | | | | | | |
| ESWGAN-GP | 24.98 ± 0.93 | 0.81 ± 0.02 | 0.72 ± 0.03 | 29.17 ± 0.76 | 0.91 ± 0.01 | 0.80 ± 0.03 |
| (DtT) (T) | | | | | | |
| ESWGAN-GP | 27.13 ± 1.00 | 0.78 ± 0.02 | 0.82 ± 0.03 | 26.06 ± 0.98 | 0.75 ± 0.02 | 0.76 ± 0.04 |
| (DpR) (S) | | | | | | |
| ESWGAN-GP | 26.61 ± 1.01 | 0.76 ± 0.02 | 0.74 ± 0.03 | 27.18 ± 0.88 | 0.80 ± 0.02 | 0.75 ± 0.03 |
| (DpT) (S) | | | | | | |
| ESWGAN-GP | 29.98 ± 0.58 | 0.84 ± 0.00 | 0.84 ± 0.02 | 27.30 ± 0.70 | 0.80 ± 0.02 | 0.82 ± 0.03 |
| (DpR) (T) | | | | | | |
| ESWGAN-GP | 29.65 ± 0.45 | 0.83 ± 0.02 | 0.78 ± 0.02 | 28.84 ± 0.57 | 0.85 ± 0.00 | 0.81 ± 0.02 |
| (DpT) (T) | | | | | | |

As expected, models trained and evaluated on the same anatomical site-such as the ESWGAN-GP trained on Distal Radius (“DtR”) and tested on the same site demonstrated superior performance, with higher PSNR values, compared to scenarios where the model was tested on a different site, such as the Distal Tibia. Interestingly, when the ESWGAN-GP model trained on Distal Tibia (ESWGAN-GP (“DtT”)) was tested on Distal Radius, it yielded relatively high PSNR and SSIM values (28.60 and 0.83, respectively), although the VIF score was lower (0.69). The output in the target domain still falls within the expected behavior, wherein cross-site testing on the Distal Radius results in lower performance metrics compared to same-site testing (24.98 vs. 29.23 in PSNR). The first two rows of FIG. 8 also exhibit the expected behavior where cross-testing performance is lower. Specifically, in Distal Radius the cortical breaks are better resolved in ESWGAN-GP (“DtR”) than in ESWGAN-GP (“DtT”). However, ESWGAN-GP (“DtR”), and ESWGAN-GP (“DtT”) both can marginally resolve cortical streaks in Distal Tibia. In both cases, ESWGAN-GP effectively does the deblurring task.

TABLE V
Segmentation performance metrics across four anatomical sites for different models evaluated on unseen source data.

| | Distal Radius | | | Diaphyseal Radius | | | Distal Tibia | | | Diaphyseal Tibia | | |
|---|---|---|---|---|---|---|---|---|---|---|---|---|
| | Dice | Jaccard | Hausdorff | Dice | Jaccard | Hausdorff | Dice | Jaccard | | Dice | Jaccard | Hausdorff |
| | coefficient | index | dist. | coefficient | index | dist. | coefficient | index | Hausdorff | coefficient | index | dist. |
| Motion | 0.83 ± | 0.72 ± | 28.35 ± | 0.98 ± | 0.96 ± | 6.93 ± | 0.92 ± | 0.85 ± | 17.36 ± | 0.98 ± | 0.96 ± | 9.70 ± |
| corrupted | 0.10 | 0.13 | 30.02 | 0.01 | 0.02 | 4.12 | 0.07 | 0.09 | 22.58 | 0.01 | 0.02 | 4.50 |
| WGAN | 0.83 ± | 0.72 ± | 28.57 ± | 0.98 ± | 0.96 ± | 6.94 ± | 0.92 ± | 0.85 ± | 17.32 ± | 0.98 ± | 0.96 ± | 9.70 ± |
| | 0.10 | 0.13 | 29.96 | 0.01 | 0.02 | 4.12 | 0.07 | 0.09 | 22.50 | 0.01 | 0.02 | 4.49 |
| WGAN- | 0.87 ± | 0.78 ± | 23.95 ± | 0.99 ± | 0.98 ± | 5.86 ± | 0.93 ± | 0.87 ± | 12.46 ± | 0.98 ± | 0.96 ± | 8.64 ± |
| GP | 0.08 | 0.11 | 25.23 | 0.00 | 0.01 | 6.33 | 0.08 | 0.07 | 15.74 | 0.01 | 0.02 | 4.50 |
| SWGAN- | 0.88 ± | 0.79 ± | 23.55 ± | 0.99 ± | 0.98 ± | 5.54 ± | 0.91 ± | 0.84 ± | 16.44 ± | 0.98 ± | 0.96 ± | 9.70 ± |
| GP | 0.08 | 0.11 | 27.00 | 0.00 | 0.01 | 4.00 | 0.06 | 0.08 | 16.49 | 0.01 | 0.02 | 4.49 |
| ESWGAN- | 0.87 ± | 0.78 ± | 24.57 ± | 0.99 ± | 0.98 ± | 5.08 ± | 0.93 ± | 0.87 ± | 15.58 ± | 0.98 ± | 0.96 ± | 9.45 ± |
| GP | 0.09 | 0.12 | 24.76 | 0.00 | 0.01 | 3.49 | 0.06 | 0.08 | 16.30 | 0.01 | 0.02 | 4.71 |

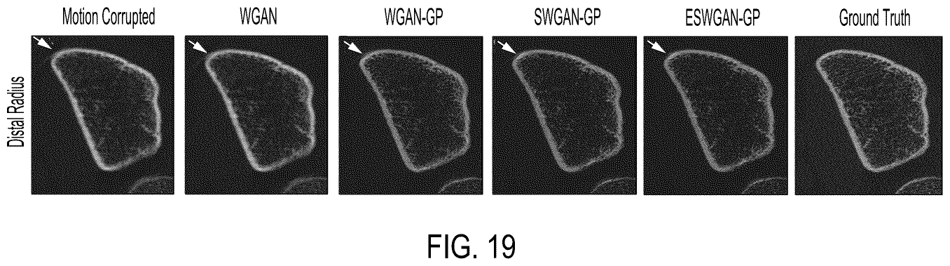

FIG. 19 shows a qualitative assessment of segmentation performance on motion-corrected images from the unseen source data generated by WGAN, WGAN-GP, SWGAN-GP, and ESWGAN-GP. The boundary denotes the cortical bone segmented by autocontour, and the arrow highlights localized boundary refinement. For optimal clarity, view this figure on a digital display.

A similar trend is observed in the diaphyseal regions, where same-site testing generally results in improved quantitative performance. An exception to this is the ESWGAN-GP (DpT) model, trained on the Diaphyseal Tibia and evaluated on the Radius, which achieves a higher PSNR than in the same-site testing (29.65 vs. 28.84) in the unseen target domain. Notably, the differences in fidelity metrics across sites are less pronounced in the diaphyseal regions compared to the distal regions. This can be attributed to the greater morphological similarity among diaphyseal sites. This observation is further supported by the last two rows of FIG. 8, where cortical streaking artifacts are more effectively mitigated in the diaphyseal regions, irrespective of the training site. While this model demonstrates modest robustness in cross-site generalization, further investigations are warranted to assess the potential of deep learning approaches that exclude specific anatomical sites during training yet retain the ability to generalize effectively across sites.

HR-pQCT is primarily valued for its ability to provide quantitative assessments, including cortical thickness, trabecular number, and bone mineral density. Accurate derivation of these metrics necessitates the segmentation of HR-pQCT images into anatomical compartments such as cortical and trabecular regions. However, motion artifacts can compromise cortical bone architecture and blur trabecular microstructures. In this study, autocontour was employed from the “ORMIR XCT” library, a segmentation process tailored to mimic the Image Processing Language (“IPL”) used by the HR-pQCT scanner, across all anatomical sites. To evaluate the impact of motion correction on segmentation performance, standard image similarity metrics were compared—Dice coefficient, Jaccard index, and Hausdorff distance—between motion-corrected and ground-truth images. The results are presented in Table V. The Dice coefficient and Jaccard index quantify the similarity between the motion-corrected segmentation and the ground truth, with higher values indicating better agreement. In contrast, the Hausdorff distance measures the maximum boundary deviation between the predicted and reference segmentations, where lower values denote improved accuracy. A uniform, empirically selected threshold was applied across all images, which may have introduced variability in the metric values across different models. FIG. 19 presents the segmentation results obtained using autocontour on motion-corrected images produced by the four models evaluated in section III-C1. A progressive refinement of the cortical boundary is observed with increasing quality of motion correction. However, due to inherent limitations of the autocontour process, a comprehensive investigation of HR-pQCT segmentation performance—both before and after motion correction—is required prior to drawing definitive conclusions.

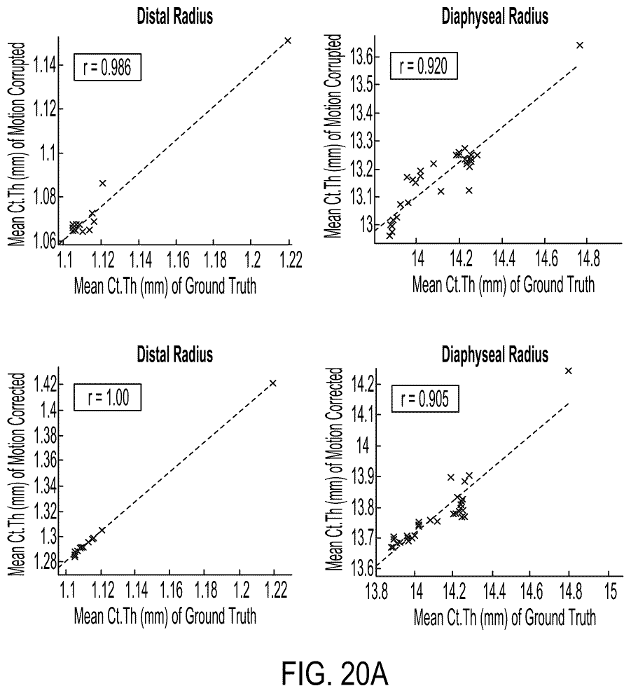

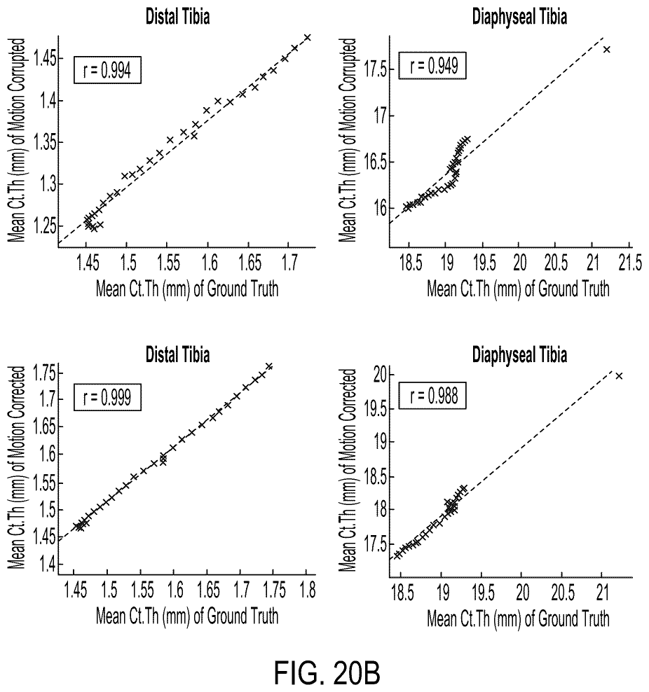

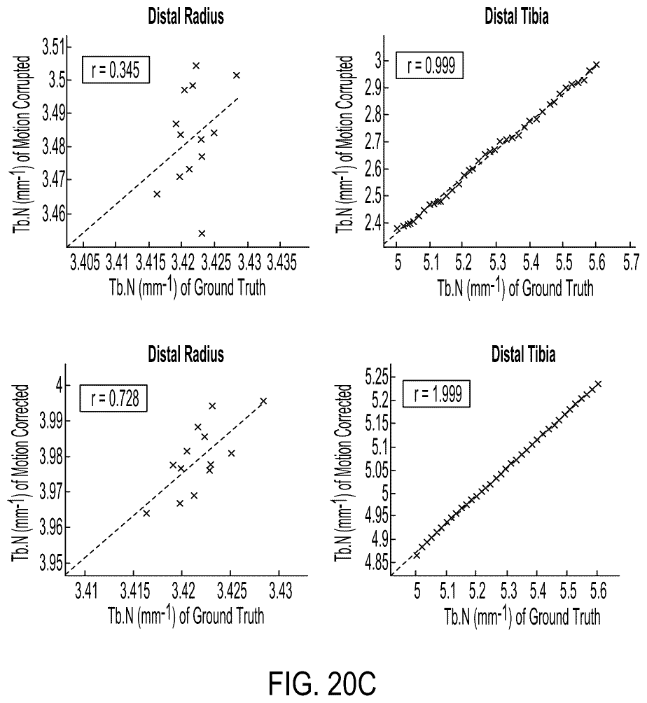

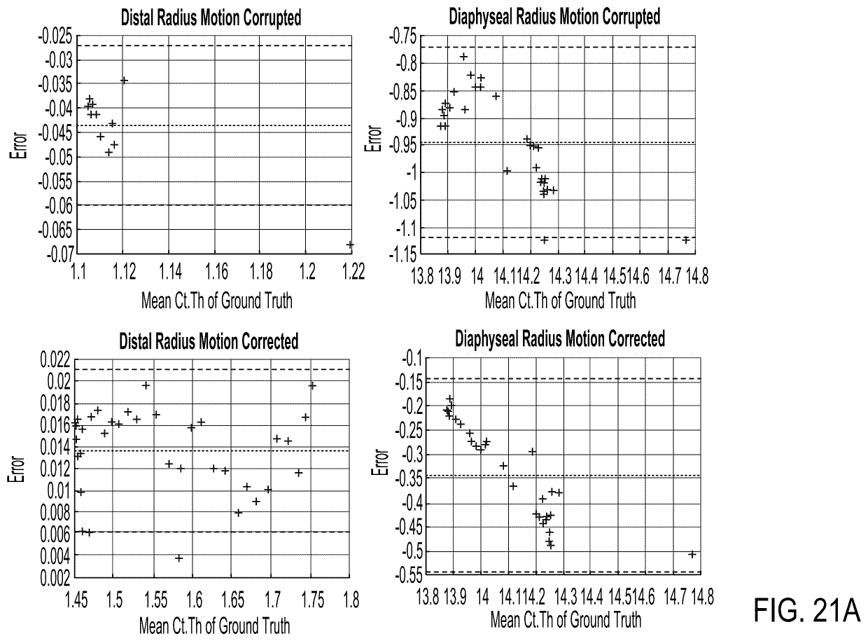

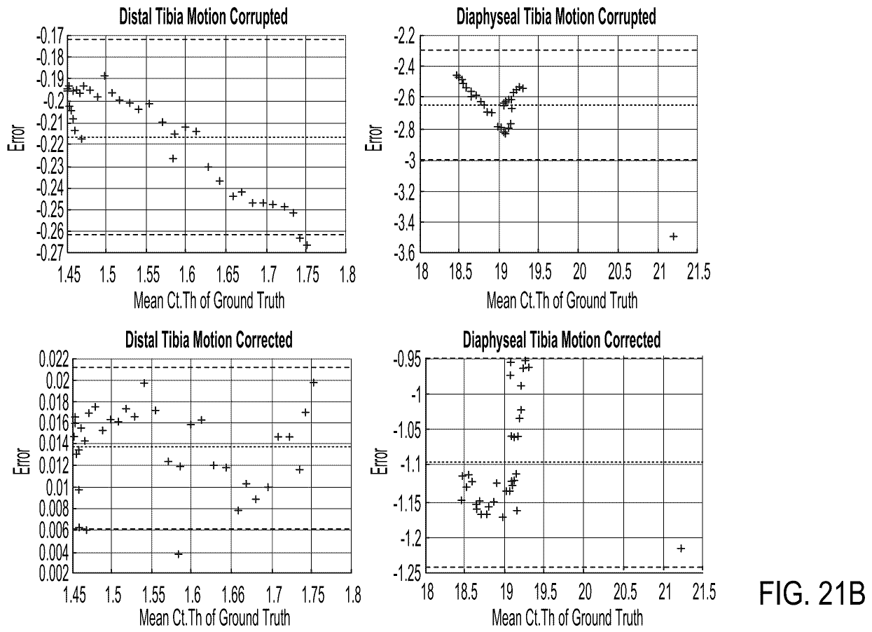

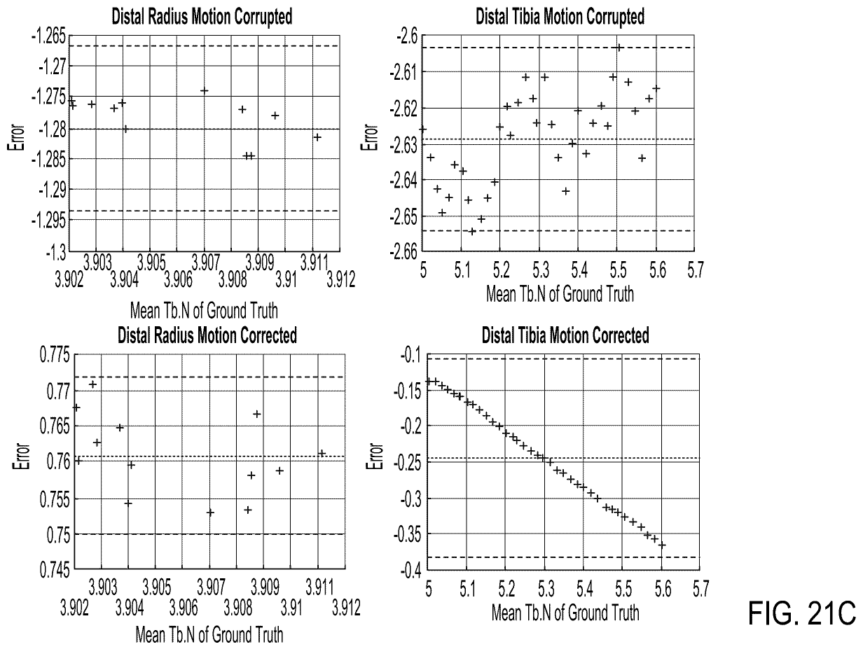

Following segmentation, in vivo quantitative assessments of bone geometry, including parameters such as cortical thickness (“Ct.Th”), trabecular number (“Tb.N”), and bone mineral density (“BMD”) can be derived from HR-pQCT imaging. These measurements have been demonstrated to capture variations associated with age, sex, disease progression, and the effects of pharmaceutical interventions. Accurate calculation of these quantitative parameters from HR-pQCT scans necessitates precise segmentation. To ensure automation and consistency across slices, autocontour and a fixed threshold were applied during the morphological operations throughout the segmentation process. This experiment was conducted on the source dataset, where ground truth measurements were available. FIGS. 20A-C demonstrate the performance of ESWGAN-GP in recovering Ct.Th in all of the four peripheral sites and Tb.N in Distal Radius and Tibia. Strong correlations were observed due to motion correction, with the exception of the Ct.Th measurement at the Diaphyseal Radius, where the lower correlation is attributed to an outlier resulting from suboptimal segmentation. Given the sensitivity of the correlation coefficient to outliers, Bland-Altman plots were presented in FIG. 21. These plots display the differences between the ground truth and the motion-corrupted measurements (first row), as well as the differences between the ground truth and motion-corrected measurements (second row), plotted against the mean of the corresponding ground truth values. A noticeable reduction in error is observed following motion correction. For instance, in the Diaphyseal Radius, the average absolute error in Ct.Th decreased from approximately 0.95 in the motion-corrupted data to 0.35 in the motion-corrected data. Similarly, in the Diaphyseal Tibia, the average error in Ct.Th was reduced from around 2.7 to 1.1. Comparable trends are evident in the distal sites for both Ct.Th and Tb.N measurements. An important observation from the Bland-Altman plots in FIG. 21 is that, although measurement errors are reduced, they do not cross zero, indicating that a complete elimination of motion-induced consequences is unlikely.

FIGS. 20A-C shows the performance of ESWGAN-GP in restoring bone geometry in the unseen source dataset. (a) and (b) illustrate the effects of motion artifacts and their correction by ESWGAN-GP on cortical thickness (“Ct.Th”) and trabecular number (“Tb.N”), respectively. The first row shows measurements from motion-corrupted data, while the second presents those after correction. The Pearson correlation coefficient (r) quantifies how well motion correction preserves the underlying trends in the measurements.

FIGS. 21A-C show Bland-Altman plots evaluating the agreement between ground truth and ESWGAN-GP-predicted bone geometry parameters relative to their mean ground truth values. (a) and (b) illustrate the effects of motion artifacts and their correction by ESWGAN-GP on cortical thickness (“Ct.Th”) and trabecular number (“Tb.N”), respectively. The first row shows measurements from motion-corrupted data, while the second presents those after correction. The red dashed line represents the mean difference, while the black dashed lines indicate the 95% limits of agreement, reflecting the expected range of variation in the prediction.

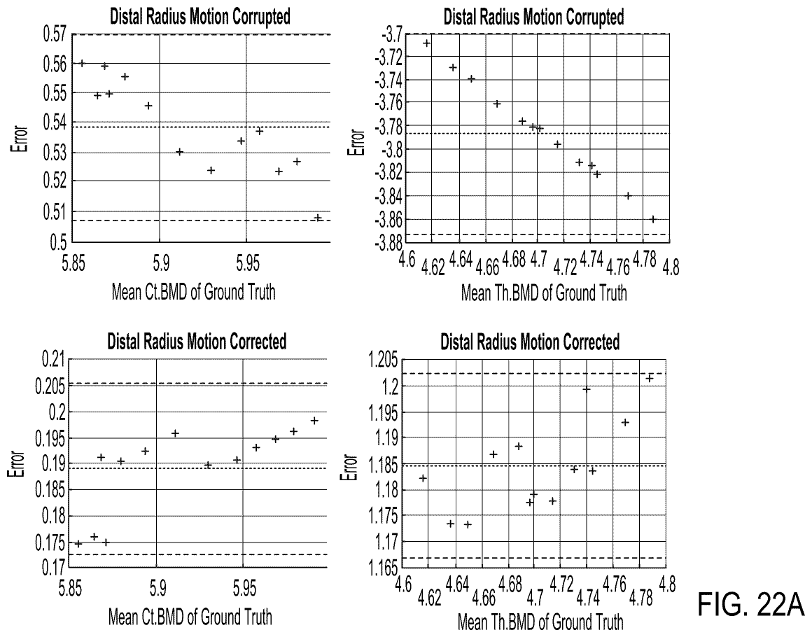

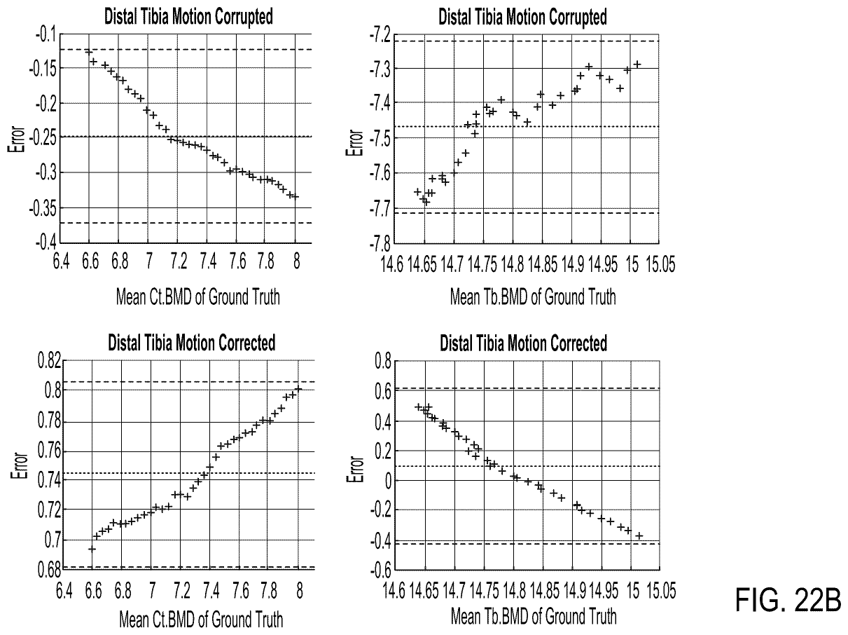

The mean bone mineral density (“BMD”) is additionally computed for both cortical (“Ct.BMD”) and trabecular (“Tb.BMD”) regions. This was achieved by applying the respective cortical and trabecular masks to the image data, followed by averaging the resulting values across the entire volume. FIGS. 22A-B present Bland-Altman plots illustrating the prediction errors of Ct.BMD and Tb.BMD relative to the ground truth BMD measurements. The analysis is conducted across both simulated corrupted and ESWGAN-GP-corrected volumes. A reduction in prediction error is observed in the motion-corrected scenarios. For example, the mean Ct.BMD in the motion-corrupted Distal Radius data is 0.54, whereas it is reduced to 0.19 following motion correction, indicating a mitigation of motion-induced artifacts. In Tb.BMD of Distal Tibia, a few volumes have nearly crossed the zero line, indicating a substantial attenuation of motion artifacts. These findings collectively suggest that while motion correction has a beneficial impact on quantitative measurements, its capacity to fully resolve motion artifacts remains uncertain and warrants further investigation through more comprehensive studies.

Notably, in FIG. 22A-B, the error appears to vary systematically with the mean BMD, demonstrating a tendency to under-estimate BMD at lower mean values and overestimate it at higher ones. This pattern may be attributable to the automated segmentation process, which tends to under-segment (i.e., thin) the cortical and trabecular compartments in regions of lower BMD and over-segment (i.e., thicken) them in regions of higher BMD. Further investigations may obtain more accurate estimations of the quantitative measurements and, consequently, enable a comprehensive evaluation of the motion correction performance.

FIGS. 22A-B show Bland-Altman plots depicting the agreement between ground truth and ESWGAN-GP-predicted values for cortical BMD (“Ct.BMD”) and trabecular BMD (“Tb.BMD”). The plots display the prediction error as a function of the mean ground truth BMD. The first row shows measurements from motion-corrupted data, while the second presents those after correction. Similar to FIGS. 21A-C, the red dashed line represents the mean difference.

As described herein, a sinogram-based approach may be used to simulate in-plane rotational motion artifacts in HR-pQCT, to train an Edge-enhanced Self-attention Wasser-stein Generative Adversarial Network with Gradient Penalty (“ESWGAN-GP”) that can effectively address motion artifacts in both simulated and real-world data. The integration of edge-enhancing skip connections, self-attention mechanisms, and a VGG-based perceptual loss contributes to the accurate reconstruction of fine microstructural features. The technology described herein employs deep learning in motion correction for HR-pQCT, potentially reducing patient rescans by up to 10% per week, a major challenge for the broader adoption of this modality. The utility of the deep learning framework, particularly the edge enhancer block, can be extended to quantitative computed tomography (“QCT”) and micro-computed tomography (“micro-CT”). The practical applicability of the proposed study is constrained by its focus on in-plane rotational motion within the simulation framework. Future work will aim to incorporate a wider range of motion artifacts, including in-plane translation, out-of-plane rotation, and z-axis translation into the simulation process, which will be subsequently utilized for training deep learning models.

### Example 2

### Motion Correction for High-Resolution Computed Tomography: Bridging the Simulation-to-Real Domain Gap

Despite advances in motion correction techniques, clinical deployment remains constrained by the substantial performance degradation when models trained on simulated motion artifacts encounter real-world motion corrupted clinical data. This simulation-to-reality gap particularly impacts high-resolution peripheral quantitative computed tomography (HR-pQCT), a critical imaging modality for microarchitectural bone assessment at peripheral anatomical sites. Patient movement during acquisition introduces artifacts that compromise the fidelity of microstructural measurements. To facilitate practical HR-pQCT motion correction research, first, HR-MoCo47K is presented, the first publicly available motion correction dataset for HR-pQCT, consisting of 38,472 simulated motion artifacts and motion-free paired images from 229 distinct patients (source domain), as well as 9,072 real-world motion corrupted images from 54 patients (target domain). Second, DuCyCADA (“Dual Cycle Consistent Adversarial Domain Adaptation”) is proposed, an unsupervised framework that mitigates the source-target distribution mismatch through dual complementary cycles: one maintaining cycle consistency while the other performs adversarial alignment via KL divergence minimization. Building on the domain-adapted representations, SWINDyT (Shifted WINdow transformer with Dynamic Tanh) is trained in an adversarial fashion on target adapted source images and their clean motion-free counterparts, enabling robust artifact suppression on real-world motion-degraded scans. The present disclosure demonstrates the application of CycleGAN-based unsupervised domain adaptation for high-resolution bone microstructure recovery, tackling the challenging problem of generating anatomically faithful reconstructions from motion-degraded inputs.

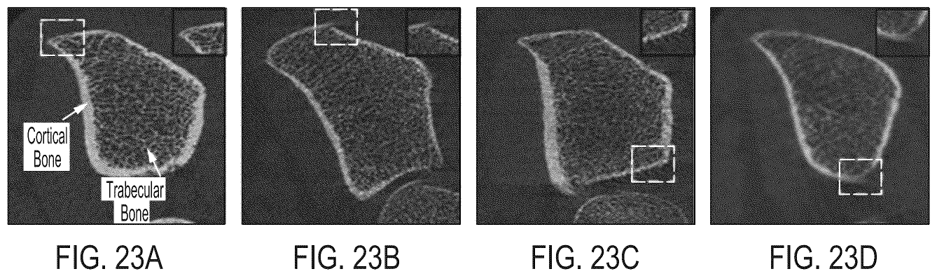

High-resolution peripheral quantitative computed tomography (“HR-pQCT”) is a low-dose (3-5 μSv), high-resolution 3D X-ray-based modality that enables in vivo quantification of bone microarchitecture, providing morphological measurements such as cortical and trabecular thickness at peripheral skeletal sites (e.g., distal radius and distal tibia), which are commonly used for the diagnosis and therapeutic monitoring of osteoporosis. Despite the use of restraining protocols, high-resolution imaging remains highly susceptible to rigid motion artifacts due to its relatively long acquisition time (e.g., 2.8 min), often resulting in cortical streaking, disruption in continuity, and trabecular smearing, as illustrated in FIG. 23(b, c).

FIG. 23A-D show an overview of motion artifacts in HR-pQCT scans of the distal radius. (a) Motion-free image with clearly defined cortical and trabecular structures; (b) disruption of cortical contiguity and streaking artifacts; (c) trabecular smearing due to motion corruption. Each image is from a different subject. Zoomed-in views of regions affected by motion artifacts (blue dashed rectangles) are shown in the top-right corner of each pane.

The current standard approach for mitigating motion artifacts involves re-scanning the patient; however, this strategy has proven to be largely ineffective, despite requiring substantial human effort. Involuntary movements such as twitches and spasms, as well as the challenges associated with imaging pediatric populations, often prevent the acquisition of artifact-free images even after repeated scans. The manufacturer has provided a visual grading system (VGS) to quantify the severity of motion artifacts, ranging from 1 to 5. A score of 1 indicates an image free from visible motion artifacts, whereas a score of 5 corresponds to severe degradation, characterized by pronounced cortical streaking, loss of cortical bone continuity, horizontal streaks, and trabecular smearing. Given the inherent subjectivity of operator-based assessments, efforts have been made to develop automated or semi-automated approaches for motion quantification. Previous approaches propose a method that estimates motion by computing the difference between parallelized projections acquired at 0° and 180°, which should be matched exactly if there were no motion. Other approaches proposed an automatic motion quantification approach using Helgason-Ludwig Consistency Conditions (“HLCC”) to quantify the deviation in scans caused by in-plane rotation and translation. Still other approaches extended previous work by integrating z-axis translation into the HLCC framework, enabling more accurate quantification of motion artifacts and their effects on morphological parameters such as cortical thickness (“Ct.Th”), trabecular thickness (“Tb.Th”), and trabecular number (“Tb.N”). While these studies focused on quantifying motion artifacts, they simulated motion during the process, thereby laying the groundwork for future motion correction. Approaches. Recently, deep neural networks have been developed to classify motion into five distinct Visual Grading Scores (“VGS”) in an effort to minimize errors stemming from operator subjectivity. However, progress in motion correction has been relatively limited, primarily due to the lack of a practical method for generating large-scale paired datasets with motion artifacts derived from high-quality (VGS score 1) images. To address this challenge, some have proposed introducing a motion blurring approach that synthetically generates motion-corrupted images from ground truth VGS score 1 data. These paired datasets are then utilized to train a CycleGAN architecture for correcting motion artifacts. Since cortical streaks, discontinuities, and trabecular smearing arise from patient motion—distinct from conventional motion blur—the proposed method lacks practical applicability. More recently, a comprehensive in-plane patient motion simulation and a deep learning-based correction method has been proposed. Specifically, the authors extended the application of HLCC conditions to simulate in-plane rotational motion on ground truth images, thereby generating a large-scale dataset for training a Generative Adversarial Network (GAN). They further enhanced the network by incorporating self-attention mechanisms and an edge enhancement module to preserve microstructures better. However, the model exhibits suboptimal performance when applied to real-world motion-corrupted data. This is primarily due to the simulation strategy, where motion corruption was modeled as a combination of in-plane motion and image blurring, introduced via reduced iterations in filtered back projection. While added blurring helps separate domains and learn the mapping, it creates a gap since real-world motion artifacts are granular rather than smooth as shown in FIG. 23(d), limiting model generalizability. Furthermore, real-world motion artifacts are inherently stochastic and patient-specific, producing degradation patterns whose full diversity cannot be captured by simulation-based augmentation strategies.

Therefore, DuCyCADA, a Dual Cycle-Consistent Adversarial Domain Adaptation framework, is proposed that bridges the distribution gap between synthetically corrupted and real-world motion-degraded scans. DuCyCADA employs two complementary CycleGANs: the first learns a supervised mapping from motion-corrupted to artifact-free images in the source domain, while the second enforces feature-level alignment through Kullback-Leibler (“KL”) divergence minimization. For artifact correction on real scans, SWINDyT is introduced, a shifted window Transformer augmented with Dynamic Tanh activations, trained adversarially to map the target-adapted source representations to their clean counterparts, thereby generalizing effectively to authentic motion-corrupted images.

Example contributions provided by this disclosure include a presentation of HR-MoCo47k, the first publicly available HR-pQCT motion correction dataset, comprising 229 pairs of simulated motion-corrupted scans with corresponding ground-truths and 54 volumes of real-world motion-corrupted scans, totaling 47,544 images. Another contribution includes, along with leveraging this dataset, DuCyCADA is introduced, a Dual Cycle-Consistent Adversarial Domain Adaptation framework that incorporates a KL-divergence based domain alignment objective. Unlike CyCADA, this approach employs a backward generator to learn pseudo-labels from real-world motion corrupted scans, which project source-domain data into the target domain. Another contribution includes a SWINDyT (Shifted WINdow Transformer with Dynamic Tanh), a supervised architecture trained on target-like source data with corresponding ground truth, designed to effectively correct motion artifacts in real-world scans. Yet another contribution includes extensive benchmarking on HR-MoCo47k establishes strong baselines, paving the way for future research in motion correction for high-resolution CT and domain adaptive medical image reconstruction.

TABLE VI
Summary of the HR-MoCo47K dataset. VGS score 1 indicates no motion
artifacts, while VGS >1 indicates the presence of motion artifacts

| Region | Patients | Images | VGS Score |
|---|---|---|---|
| Distal Radius | 103 | 17,304 | 1 (no artifacts) |
| Distal Tibia | 126 | 21,168 | 1 (no artifacts) |
| Distal Radius | 40 | 6,720 | >1 (with artifacts) |
| Distal Tibia | 14 | 2,352 | >1 (with artifacts) |
| Total | 283 | 47,544 | — |

3. HR-MoCo47K Dataset: 3.1. Data Collection. HR-pQCT data were collected through the Function, Imaging, and Testing (“FIT”) Resource Core at the Indiana Center for Musculoskeletal Health. The dataset comprises scans from 283 patients imaged between 2018 and 2022 under an IRB approved by Indiana University. Informed consent was ensured from all the participants. Scans were acquired using XtremeCT II (Scanco Medical, Brüttisellen, Switzerland) on the non-dominant arm, a.k.a., distal radius (4% from radius reference line), and the contralateral leg, a.k.a., distal tibia (7.3% from tibia reference line). Imaging was performed at 68 kVp and 1.47 mA, yielding 168 slices (10.2 mm) with an isotropic voxel size of 60.7 μm.

VGS score was graded by a trained operator ranging from 1 (artifact-free) to 5 (severe degradations in cortical and trabecular microarchitectures). Among 283 patients, 103 contributed distal radius scans (17,304 images) and 126 contributed distal tibia scans (21,168 images). All of these scans are graded with a VGS score of 1, indicating no motion artifacts. In addition, the dataset includes 40 distal radius scans and 14 distal tibia scans with VGS scores greater than 1, reflecting varying degrees of motion artifacts. Together, these collections constitute the HR-MoCo47K dataset. The dataset is summarized in Table 1.

The problem is formulated within the framework of unsupervised domain adaptation, where the source image, xs∈ [symbol] ², represents motion-corrupted simulated data derived from the ground truth image, ys∈ [symbol] ². Additionally, target domain data, xt∈ [symbol] ², is provided corresponding to real-world motion-corrupted images; however, the labels, yt∈ [symbol] ², for this domain are unavailable.

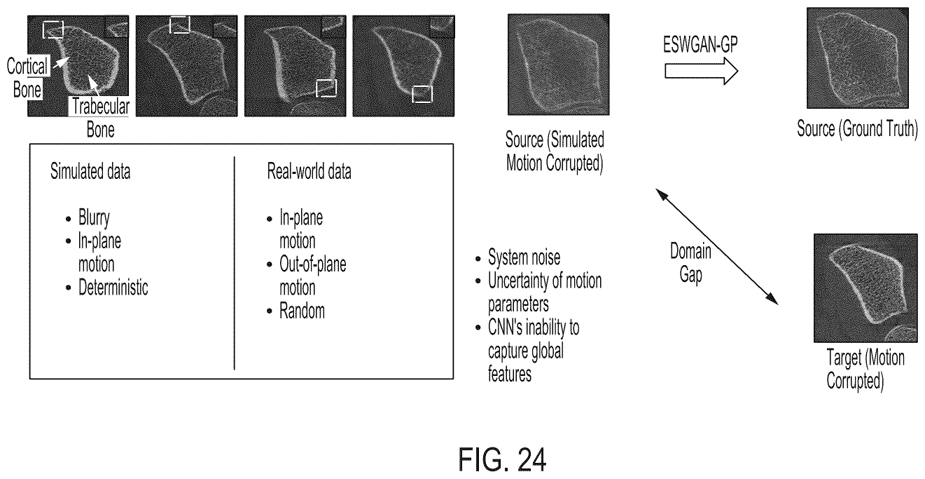

FIG. 24 shows a schematic illustration demonstrating the domain gap between the simulated motion corruption and the target motion corruption.

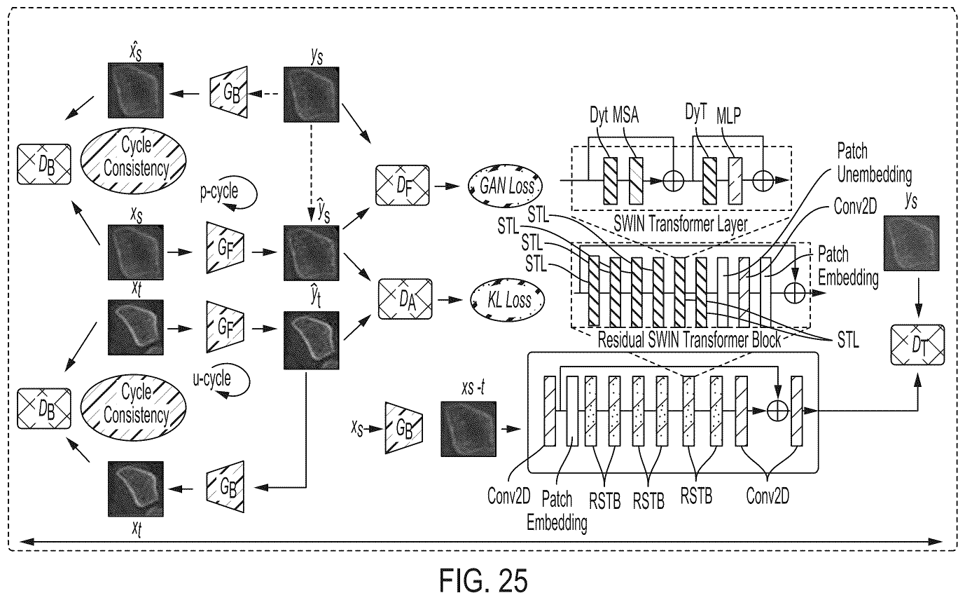

Two generator networks are utilized, GF and GB, which are responsible for translating motion-corrupted images to their motion-corrected counterparts and performing the inverse mapping, respectively. As shown in FIG. 25, in the first paired CycleGAN, the generator GF maps the input xs to a predicted motion-corrected image ŷs=GF(xs), which is then adversarially optimized to be indistinguishable from the corresponding ground truth ys. This constitutes the following objective function for the paired GAN:

$${ℒ}_{pGAN} ( {G}_{F} , {D}_{F} , {X}_{S} , {Y}_{S} ) = {𝔼}_{{y}_{s} \cdot {Y}_{s}} [ {( {D}_{F} ( {Y}_{S} ) - 1 )}^{2} ] + {𝔼}_{{x}_{s} ∼ {X}_{s}} [ {( {D}_{F} ( {G}_{F} ( {x}_{S} ) ) )}^{2} ] \qquad ( 18 )$$

To enforce both pixel-level and semantic consistency, a generator GB is provided that performs the inverse mapping from ŷs to ŷxₛ and optimizes the cycle consistency through the following objective function.

$${ℒ}_{pGANi} ( {G}_{B} , {D}_{B} , {X}_{S} , {Y}_{S} ) = {𝔼}_{{x}_{s} ∼ {X}_{s}} [ {( {D}_{B} ( {x}_{S} ) - 1 )}^{2} ] + {𝔼}_{{x}_{s} ∼ {X}_{s}} [ {( {D}_{B} ( {G}_{B} ( {G}_{F} ( {x}_{S} ) ) ) )}^{2} ] \qquad ( 19 )$$

To enforce cycle consistency and constrain the generator from introducing hallucinated structures, a [symbol] 2 penalty is incorporated, shown in equation 20. This regularization can be significant in high-resolution modalities such as HR-pQCT bone imaging, where even minor hallucinations can significantly distort the fine trabecular architecture.

$${ℒ}_{pcyc} ( {G}_{F} , {G}_{B} , {X}_{S} ) = {𝔼}_{{x}_{s} ∼ {X}_{s}} [ {‖ ( {G}_{B} ( {G}_{F} ( {x}_{S} ) ) ) - {x}_{S} ‖}_{2} ] \qquad ( 20 )$$

The overall architecture of the paired GAN is designed to learn a mapping from motion-corrupted data to motion-corrected data within the source domain, while ensuring consistency with the input. This enforces structural fidelity, particularly preserving fine microstructural details. In the case of the unpaired CycleGAN, a similar strategy is employed, but in the reverse direction. Specifically, the motion-corrupted image from the target domain, denoted as xt, is passed through the forward generator GF to produce a synthetic motion-corrected image ŷt, which serves as a pseudo-label. Subsequently, the backward generator GB is utilized, which maps ŷt back to a reconstructed image ŷxₜ, for which there is a reference label xt. The following objective function is optimized during the training.

$${ℒ}_{uGANi} ( {G}_{F} , {G}_{B} , {D}_{B} , {X}_{t} ) = {𝔼}_{{x}_{t} ∼ {X}_{t}} [ {( {D}_{B} ( {x}_{t} ) - 1 )}^{2} + {𝔼}_{{x}_{t} ∼ {X}_{t}} [ {( {D}_{B} ( {G}_{B} ( {G}_{F} ( {x}_{t} ) ) ) )}^{2} ] \qquad ( 21 )$$

Likewise, cycle consistency is enforced with the pseudo label and the reference label through the following loss function

$${ℒ}_{ucyc} ( {G}_{F} , {G}_{B} , {X}_{t} ) = {𝔼}_{{x}_{s} ∼ {X}_{s}} [ {‖ ( {G}_{B} ( {\hat{y}}_{t} ) ) - {x}_{t} ‖}_{2} ] \qquad ( 22 )$$

In contrast to prior domain adaptation strategies, which rely on direct unpaired translation between xs and xt, a different approach is adopted by directly aligning ŷs and ŷt in the probability space. Specifically, the generator GF maps xs and xt into their feature representations, ŷs and ŷt. These representations are then trained adversarially against a domain-adaptive discriminator DA, which employs a Kullback-Leibler (KL) divergence loss to reduce the domain gap-without explicitly transforming the inputs into a latent space.

$${ℒ}_{cyc - KL} = {𝔼}_{{x}_{s} ∼ {X}_{s}} [ log ( \frac{0.5}{f ( {D}_{A} ( {G}_{F} ( {x}_{t} ) ) )} ) ] + {𝔼}_{{x}_{t} ∼ {X}_{t}} [ log ( \frac{1}{f ( {D}_{A} ( {G}_{F} ( {x}_{t} ) ) )} ) ] \qquad ( 23 )$$

In this context, f denotes a probabilistic mapping function, such as the logarithmic softmax, which transforms input values into a probability distribution. Lastly, the loss function is augmented with both identity and total variation terms, which have been shown to effectively suppress unwanted artifacts across a wide range of applications.

$${ℒ}_{IDT} ( {G}_{F} , {G}_{B} ) = {𝔼}_{{y}_{s} ∼ {Y}_{s}} [ {‖ {G}_{F} ( {y}_{s} ) - {y}_{s} ‖}_{2} ] + {𝔼}_{{x}_{t} ∼ {X}_{t}} [ {‖ {G}_{B} ( {x}_{t} ) - {x}_{t} ‖}_{2} ] \qquad ( 24 )$$

$${{ℒ}_{TV} ( {G}_{F} ) = ‖ {G}_{F (} {x}_{s} ) ‖}_{TV} + {‖ {G}_{F} ( {x}_{t} ) ‖}_{TV} \qquad ( 25 )$$

The overall objective function can be written as:

$${ℒ}_{DuCyCADA} = {λ}_{1} {ℒ}_{pGAN} + {λ}_{2} {ℒ}_{pGANi} + {λ}_{3} {ℒ}_{pcyc} + {λ}_{4} {ℒ}_{uGANi} + {λ}_{5} {ℒ}_{ucyc} + {λ}_{6} {ℒ}_{cyc - KL} + {λ}_{7} ℒ IDT + {λ}_{8} {ℒ}_{TV} \qquad ( 26 )$$

FIG. 25 shows an architecture of DuCYCADA and SWINDyT:DuCyCADA employs dual generators. The forward generator GF maps simulated motion-corrupted images xs to corrected outputs ŷs with adversarial supervision from discriminator DF, while unpaired real motion-corrupted data xt is processed through GF to produce pseudo-corrected ŷt. Domain alignment between ŷs and ŷt is enforced via KL divergence with discriminator DA. The backward generator GB completes the cycle, with cycle consistency losses preserving structural fidelity in both cycles. The target-adapted source images xs→t are then processed by SWINDyT alongside ground truth ys for robust HR-pQCT motion correction.

4.2 Motion Correction. After performing domain adaptation, where the motion-corrupted source images xₛ ∈ [symbol] ^{H}^{_{×}}^{W}^{_{×}}^{e} are transformed with the trained GB to align with the target domain distribution, yielding xₛ₋ₜ∈ [symbol] ^{H}^{_{×}}^{WN}^{_{×}}^{c}, the adapted data is fed into the SWINDyT network, T_{F}, to perform motion correction within the target domain. In this framework, SWINDyT serves as the generator, while a U-Net-based discriminator is employed to distinguish between the adapted input xₛ₋ₜ and the corresponding ground truth image yₛ.

4.2.1 Shifted Windows Transformer with Dynamic Tanh (“SWINDyT”). To leverage convolutional inductive biases, xₛ₋ₜ is first processed through a convolutional layer and subsequently tokenized to obtain a feature sequence f∈ [symbol] ^{HW}^{_{×}}^{c}. The tokenized feature sequence is subsequently processed through a series of Residual Swin Transformer Blocks (“RSTBs”). Within the i^{th} RSTB, the initial feature representation fi,0 is propagated through N Swin Transformer Layers (“STLs”), producing a sequence of intermediate feature representations denoted as fi,1, fi,2, . . . , fi,N, as shown in Equation 27.

$${f}_{i , j} = {STL}_{i , j} ( {f}_{i , j - 1} ) , j = 1 , 2 , … , N \qquad ( 27 )$$

The output of the final STL is passed through a patch un-embedding module to transform the feature representation from the token domain back to the spatial domain. This spatial feature is then processed by a convolutional layer to refine local representations. Subsequently, the processed feature is re-embedded into patch tokens using a patch embedding layer. To facilitate effective gradient flow and preserve low-level information, a residual skip connection is employed, linking the re-embedded feature with the original input token, shown in Equation 28.

$${f}_{i , out} = CONV 2 {D}_{i} ( {f}_{i , N} ) + {f}_{i , 0} \qquad ( 28 )$$

Each STL incorporates a standard multi-head self-attention mechanism. To enhance local contextual modeling, a shifted window-based attention strategy is applied in alternating layers, enabling cross-window interactions.

4.2.2 SWIN Transformer Layer (STL). Each STL layer receives an input feature fi,j∈ [symbol] ^{HW}^{_{×}}^{c}, which is reshaped into (HW/M²)×C×M² by partitioning the spatial domain into non-overlapping windows of size M×M. Within each window, attention is computed via query (Q), key (K), and value (V) matrices as follows:

$$Attention ( Q , K , V ) = SoftMax ( \frac{{QK}^{T}}{\sqrt{d}} + B ) V \qquad ( 29 )$$

In this architecture, the query, key, and value matrices, denoted as Q, K, V∈ [symbol] ^{M2}^{_{×}}^{d}, are computed by applying linear projections to the input features using learnable matrices P^{ö}, P^{K}, and P^{V}, respectively. These projection matrices are shared across all local windows to promote parameter efficiency and ensure consistent feature representation. Multi-head self-attention (“MSA”) is employed to capture diverse contextual dependencies by computing attention in parallel across multiple heads. To facilitate cross-window information flow and enhance representational capacity, the model incorporates shifted window-based multi-head self-attention (“SW-MSA”), wherein the spatial configuration of attention windows is cyclically shifted by an off-set between consecutive transformer blocks. The resulting representations are subsequently processed by the Dynamic Tanh (“DyT”) activation module, followed by a multi-layer perceptron (“MLP”) with GELU activation, introducing non-linearity and further enriching the feature representations. The layer normalization was replaced with DyT, which has demonstrated effectiveness in stability for both vision and language tasks. Unlike conventional normalization techniques, DyT constrains extreme activation values using an S-shaped hyperbolic tangent function modulated by a learnable scaling parameter a, thereby stabilizing training dynamics without explicit normalization.

4.2.3 Loss functions. The proposed objective function is closely aligned with domain adaptation frameworks, albeit without the incorporation of cycle-consistency. Empirical evidence suggests that enforcing cycle-consistency constraints impairs the generative capacity of the model, ultimately degrading its performance in correcting motion artifacts. The following objective function is utilized for motion correction.

$${ℒ}_{GAN} ( {T}_{F} , {G}_{B} , {D}_{T} , {X}_{S} , {Y}_{S} ) = {𝔼}_{{y}_{s} ∼ {Y}_{s}} [ {( {D}_{T} ( {y}_{s} ) - 1 )}^{2} ] + {𝔼}_{{x}_{s} ∼ {X}_{s}} [ {( {D}_{T} ( {T}_{F} ( {G}_{B} ( {x}_{s} ) ) ) )}^{2} ] \qquad ( 30 )$$

To stabilize training, consistency loss, TV loss, and VGG-based content loss are also utilized.

$${ℒ}_{Consistency} ( TF ) = {𝔼}_{{y}_{s} ∼ {Y}_{s}} [ {‖ {T}_{F} ( {x}_{s - t} ) - {y}_{s} ‖}_{1} ] \qquad ( 31 )$$

$${ℒ}_{JST} ( {T}_{F} ) = {‖ {T}_{F} ( {x}_{s - t} ) ‖}_{T V} \qquad ( 32 )$$

$${ℒ}_{Consistency} ( {T}_{F} ) = {‖ VGG ( {T}_{F} ( {x}_{s - t} ) ) - VGG ( {y}_{s} ) ‖}_{1} \qquad ( 33 )$$

Together, these components constitute the first deep learning-based framework designed to effectively mitigate motion artifacts in real-world HR-pQCT data.
5. Experimental Design. Networks were trained on a single NVIDIA RTX A5500 GPU (24 GB) and conducted experiments on the dataset described in Section 3. In DuCYCADA, a ResNet-based generator was used and a vanilla VGG-style discriminator, following CycleGAN. Models were optimized with Adam, starting from a learning rate of 8×10−5, reduced by a factor of 0.5 on plateau. To assess domain alignment, feature distributions were visualized via t-SNE on 100 randomly sampled images and compared against CyCADA. For motion correction, SWINDyT was employed as the generator and a U-Net-style discriminator, trained with Adam at a fixed learning rate of 8×10−5. Performance was benchmarked against ESWGAN-GP, GAN-CIRCLE, SWINIR, and the unsupervised domain-adaptive framework UDA-SR.

Since the test data is unpaired and unsupervised, both reference-based metrics-PSNR, SSIM, VIF, and LPIPS (AlexNet-trained)—are reported and no-reference predictors, including BRISQUE, NIQE, and CLIPQA, to comprehensively evaluate both perceptual fidelity and statistical image quality.

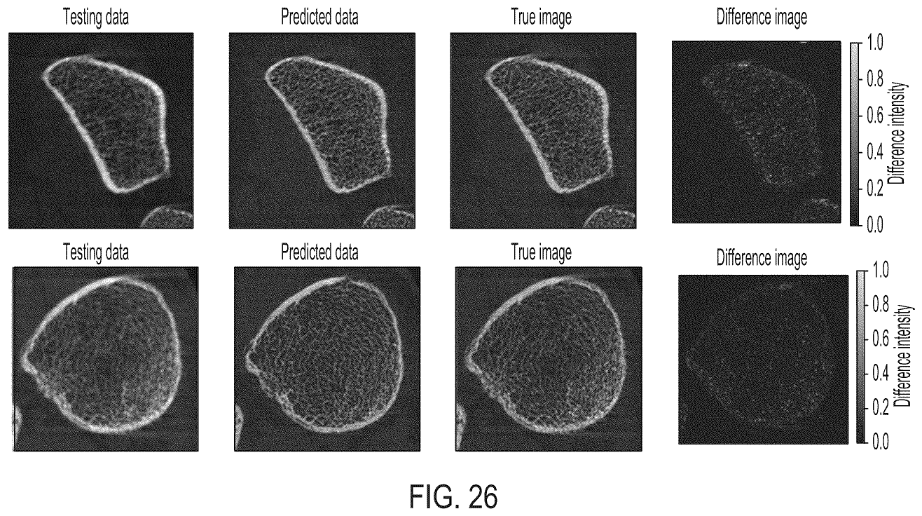

FIG. 26 shows top row displays images of the distal radius, while the bottom row depicts images of the distal tibia. The testing data were processed through the domain-adaptive network, followed by the transformer-based motion correction module (middle column). As the data were acquired from a real-world scanner, ground truth images are unavailable; however, the corrected outputs demonstrate qualitative improvements over the original real-world images.

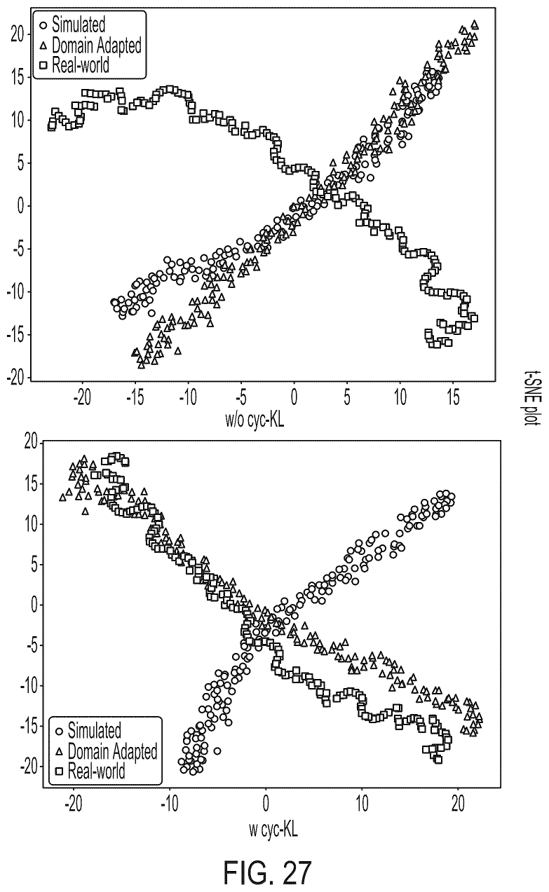

FIG. 27 shows two T-distributed stochastic neighbor embedding plots. t-SNE visualization compares the final layer representations with and without the domain adaptation piece. In the top plot, the absence of domain adaptation results in simulated artifacts and the adapted artifacts occupying the same embedding space. In contrast, the bottom figure demonstrates that the domain adaptation piece shifts the embeddings closer to real-world representations, effectively bridging the gap between simulated and real-world artifacts. t-SNE is a dimensionality reduction technique that projects a high-dimensional feature map into a two-dimensional cluster-like visualization. It is primarily used for visualization, allowing for the observation of how samples from different domains are distributed and how closely the domains align with each other in feature space.

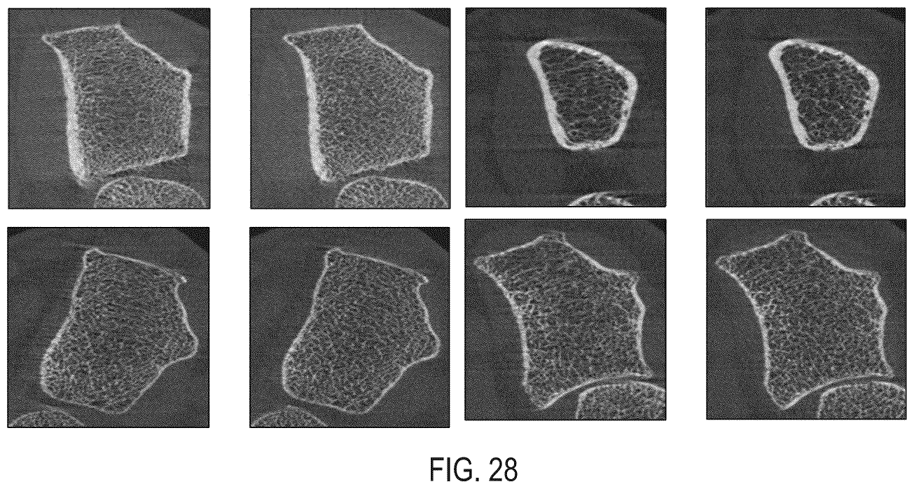

FIG. 28 shows pairs of motion corrupted images followed by the motion corrected image.

The present disclosure has described one or more preferred examples, and it should be appreciated that many equivalents, alternatives, variations, and modifications, aside from those expressly stated, are possible and within the scope of the invention.

It is to be understood that the disclosure is not limited in its application to the details of construction and the arrangement of components set forth in the accompanying description or illustrated in the accompanying drawings. The disclosure is capable of other examples and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings.

As used herein, unless otherwise limited or defined, discussion of particular directions is provided by example only, with regard to particular examples or relevant illustrations. For example, discussion of “top,” “front,” or “back” features is generally intended as a description only of the orientation of such features relative to a reference frame of a particular example or illustration. Correspondingly, for example, a “top” feature may sometimes be disposed below a “bottom” feature (and so on), in some arrangements or examples. Further, references to particular rotational or other movements (e.g., counterclockwise rotation) is generally intended as a description only of movement relative a reference frame of a particular example of illustration.

In some examples, aspects of the disclosure, including computerized implementations of methods according to the disclosure, can be implemented as a system, method, apparatus, or article of manufacture using standard programming or engineering techniques to produce software, firmware, hardware, or any combination thereof to control a processor device (e.g., a serial or parallel general purpose or specialized processor chip, a single- or multi-core chip, a microprocessor, a field programmable gate array, any variety of combinations of a control unit, arithmetic logic unit, and processor register, and so on), a computer (e.g., a processor device operatively coupled to a memory), or another electronically operated controller to implement aspects detailed herein. Accordingly, for example, examples of the disclosure can be implemented as a set of instructions, tangibly embodied on a non-transitory computer-readable media, such that a processor device can implement the instructions based upon reading the instructions from the computer-readable media. Some examples of the disclosure can include (or utilize) a control device such as an automation device, a special purpose or general purpose computer including various computer hardware, software, firmware, and so on, consistent with the discussion below. As specific examples, a control device can include a processor, a microcontroller, a field-programmable gate array, a programmable logic controller, logic gates etc., and other typical components that are known in the art for implementation of appropriate functionality (e.g., memory, communication systems, power sources, user interfaces and other inputs, etc.).

The term “article of manufacture” as used herein is intended to encompass a computer program accessible from any computer-readable device, carrier (e.g., non-transitory signals), or media (e.g., non-transitory media). For example, computer-readable media can include but are not limited to magnetic storage devices (e.g., hard disk, floppy disk, magnetic strips, and so on), optical disks (e.g., compact disk (CD), digital versatile disk (DVD), and so on), smart cards, and flash memory devices (e.g., card, stick, and so on). Additionally it should be appreciated that a carrier wave can be employed to carry computer-readable electronic data such as those used in transmitting and receiving electronic mail or in accessing a network such as the Internet or a local area network (LAN). Those skilled in the art will recognize that many modifications may be made to these configurations without departing from the scope or spirit of the claimed subject matter.

Certain operations of methods according to the disclosure, or of systems executing those methods, may be represented schematically in the FIGS. or otherwise discussed herein. Unless otherwise specified or limited, representation in the FIGS. of particular operations in particular spatial order may not necessarily require those operations to be executed in a particular sequence corresponding to the particular spatial order. Correspondingly, certain operations represented in the FIGS., or otherwise disclosed herein, can be executed in different orders than are expressly illustrated or described, as appropriate for particular examples of the disclosure. Further, in some examples, certain operations can be executed in parallel, including by dedicated parallel processing devices, or separate computing devices configured to interoperate as part of a large system.

As used herein in the context of computer implementation, unless otherwise specified or limited, the terms “component,” “system,” “module,” and the like are intended to encompass part or all of computer-related systems that include hardware, software, a combination of hardware and software, or software in execution. For example, a component may be, but is not limited to being, a processor device, a process being executed (or executable) by a processor device, an object, an executable, a thread of execution, a computer program, or a computer. By way of illustration, both an application running on a computer and the computer can be a component. One or more components (or system, module, and so on) may reside within a process or thread of execution, may be localized on one computer, may be distributed between two or more computers or other processor devices, or may be included within another component (or system, module, and so on).

In some implementations, devices or systems disclosed herein can be utilized or installed using methods embodying aspects of the disclosure. Correspondingly, description herein of particular features, capabilities, or intended purposes of a device or system is generally intended to inherently include disclosure of a method of using such features for the intended purposes, a method of implementing such capabilities, and a method of installing disclosed (or otherwise known) components to support these purposes or capabilities. Similarly, unless otherwise indicated or limited, discussion herein of any method of manufacturing or using a particular device or system, including installing the device or system, is intended to inherently include disclosure, as examples of the disclosure, of the utilized features and implemented capabilities of such device or system.

In some examples, any suitable computer-readable media can be used for storing instructions for performing the functions and/or processes described herein. For example, in some examples, computer-readable media can be transitory or non-transitory. For example, non-transitory computer-readable media can include media such as magnetic media (e.g., hard disks, floppy disks), optical media (e.g., compact discs, digital video discs, Blu-ray discs), semiconductor media (e.g., RAM, flash memory, EPROM, EEPROM), any suitable media that is not fleeting or devoid of any semblance of permanence during transmission, and/or any suitable tangible media. As another example, transitory computer-readable media can include signals on networks, in wires, conductors, optical fibers, circuits, or any suitable media that is fleeting and devoid of any semblance of permanence during transmission, and/or any suitable intangible media.

In some implementations, devices or systems disclosed herein can be utilized or installed using methods embodying aspects of the disclosure. Correspondingly, description herein of particular features, capabilities, or intended purposes of a device or system is generally intended to inherently include disclosure of a method of using such features for the intended purposes, a method of implementing such capabilities, and a method of installing disclosed (or otherwise known) components to support these purposes or capabilities. Similarly, unless otherwise indicated or limited, discussion herein of any method of manufacturing or using a particular device or system, including installing the device or system, is intended to inherently include disclosure, as examples of the disclosure, of the utilized features and implemented capabilities of such device or system.

As used herein, unless otherwise defined or limited, ordinal numbers are used herein for convenience of reference based generally on the order in which particular components are presented for the relevant part of the disclosure. In this regard, for example, designations such as “first,” “second,” etc., generally indicate only the order in which the relevant component is introduced for discussion and generally do not indicate or require a particular spatial arrangement, functional or structural primacy or order.

As used herein, unless otherwise defined or limited, directional terms are used for convenience of reference for discussion of particular figures or examples. For example, references to downward (or other) directions or top (or other) positions may be used to discuss aspects of a particular example or figure, but do not necessarily require similar orientation or geometry in all installations or configurations.

This discussion is presented to enable a person skilled in the art to make and use examples of the disclosure. Various modifications to the illustrated examples will be readily apparent to those skilled in the art, and the generic principles herein can be applied to other examples and applications without departing from the principles disclosed herein. Thus, examples of the disclosure are not intended to be limited to examples shown, but are to be accorded the widest scope consistent with the principles and features disclosed herein and the claims below. The accompanying detailed description is to be read with reference to the figures, in which like elements in different figures have like reference numerals. The figures, which are not necessarily to scale, depict selected examples and are not intended to limit the scope of the disclosure. Skilled artisans will recognize the examples provided herein have many useful alternatives and fall within the scope of the disclosure.

Also as used herein, unless otherwise limited or defined, “or” indicates a non-exclusive list of components or operations that can be present in any variety of combinations, rather than an exclusive list of components that can be present only as alternatives to each other. For example, a list of “A, B, or C” indicates options of: A; B; C; A and B; A and C; B and C; and A, B, and C. Correspondingly, the term “or” as used herein is intended to indicate exclusive alternatives only when preceded by terms of exclusivity, such as “either,” “one of,” “only one of,” or “exactly one of.” Further, a list preceded by “one or more” (and variations thereon) and including “or” to separate listed elements indicates options of one or more of any or all of the listed elements. For example, the phrases “one or more of A, B, or C” and “at least one of A, B, or C” indicate options of: one or more A; one or more B; one or more C; one or more A and one or more B; one or more B and one or more C; one or more A and one or more C; and one or more of each of A, B, and C. Similarly, a list preceded by “a plurality of” (and variations thereon) and including “or” to separate listed elements indicates options of multiple instances of any or all of the listed elements. For example, the phrases “a plurality of A, B, or C” and “two or more of A, B, or C” indicate options of: A and B; B and C; A and C; and A, B, and C. In general, the term “or” as used herein only indicates exclusive alternatives (e.g. “one or the other but not both”) when preceded by terms of exclusivity, such as “either,” “one of,” “only one of,” or “exactly one of.”

Also as used herein, unless otherwise specified or limited, the terms “about” and “approximately,” as used herein with respect to a reference value, refer to variations from the reference value of ±15% or less (e.g., ±10%, ±5%, etc.), inclusive of the endpoints of the range. Similarly, the term “substantially equal” (and the like) as used herein with respect to a reference value refers to variations from the reference value of less than ±30% (e.g., ±20%, ±10%, ±5%) inclusive. Where specified, “substantially” can indicate in particular a variation in one numerical direction relative to a reference value. For example, “substantially less” than a reference value (and the like) indicates a value that is reduced from the reference value by 30% or more, and “substantially more” than a reference value (and the like) indicates a value that is increased from the reference value by 30% or more.

It will be appreciated by those skilled in the art that while the invention has been described above in connection with particular examples and examples, the invention is not necessarily so limited, and that numerous other examples, examples, uses, modifications and departures from the examples, examples and uses are intended to be encompassed by the claims attached hereto. The entire disclosure of each patent and publication cited herein is incorporated by reference, as if each such patent or publication were individually incorporated by reference herein. Various features and advantages of the invention are set forth in the following claims.

Various features and advantages of the disclosure are set forth in the following claims.

1. An imaging system:
a computing device being configured to:
receive a motion corrupted image of a portion of a subject;
provide the motion corrupted image to a first machine learning model, the first machine learning model being trained to correct motion corruption, the first machine learning model having been trained by using a plurality of training image pairs, wherein each training image pair of the plurality of training image pairs includes:
a first image; and
a second image being a motion corrupted version of the first image,
wherein the motion corruption in the second image is simulated; and
receive, from the first machine learning model, a corrected version of the motion corrupted image.

2. The imaging system of claim 1, wherein the computing device is further configured to construct each training image pair by:
receiving the first image; and
introducing a simulation motion artifact into the first image to generate the second image.

3. The imaging system of claim 2, wherein the computing device is further configured to introduce the simulation motion artifact to each second image of each training image pair of the plurality of training image pairs by:
receiving a sinogram of the first image;
shifting one or more lines of the sinogram to generate a shifted sinogram; and
generating the second image by reconstructing the shifted sinogram.

4. The imaging system of claim 1, wherein the first machine learning model corrects at least one of an in plane translation of the portion of the subject, an in plane rotation of the portion of the subject, or a z-axis translation of the portion of the subject.

5. The imaging system of claim 1, wherein the corrected version of the motion corrupted image is a motion corrected image; and
wherein the computing device is further configured to determine, using the motion corrected image, one or more anatomical measurements of the portion of the subject.

6. The imaging system of claim 5, wherein the computing device is further configured to determine a presence or a score of a medical condition, based on the one or more anatomical measurements.

7. The imaging system of claim 6, wherein the motion corrupted image is a high-resolution peripheral quantitative computed tomography (HR-pQCT) image; and
wherein the one or more anatomical measurements include a cortical thickness, a trabecular number, or a bone mineral density; and
wherein the medical condition is osteoporosis.

8. The imaging system of claim 1, wherein each first image of the plurality of training image pairs is a motion free image.

9. The imaging system of claim 1, wherein the computing device is further configured to generate each second image of each training image pair of the plurality of training image pairs by:
introducing a simulated motion artifact into the first image to generate a simulated motion image;
providing the simulated motion image to a second machine learning model, the second machine learning model changing a first domain of the simulated motion image into a second domain; and
receiving the second image from the machine learning model.

10. The imaging system of claim 9, wherein the second machine learning model is a style transfer machine learning model or a transformer network.

11. The imaging system of claim 9, wherein the second machine learning model includes a first pair of generators and a second pair of generators.

12. The imaging system of claim 11, wherein the second machine learning model includes a first cycle generative adversarial network (CycleGAN) and a second CycleGAN; and
wherein the first CycleGAN includes the first pair of generators; and
wherein the second CycleGAN includes the second pair of generators.

13. The imaging system of claim 1, wherein the motion corruption in each second image of the plurality of training image pairs is introduced directly into a sinogram of the first image to generate each second image.

14. A method comprising:
receiving a motion corrupted image of a portion of a subject;
correcting the motion corrupted image to generate a motion corrected image by:
providing the motion corrupted image to a first machine learning model; and
receiving the motion corrected image from the first machine learning model, wherein the first machine learning model has been trained by using a plurality of training image pairs, each training image pair includes:
a first image; and
a second image, wherein the second image is a motion corrupted version of the first image.

15. The method of claim 14, further comprising generating each second image of each training image pair of the plurality of training image pairs by:
introducing a motion artifact in the first image to generate a simulated motion image;
providing the simulated motion image to a second machine learning model; and
receiving the second image from the second machine learning model.

16. The method of claim 15, wherein the second machine learning model is a transfer machine learning model or a transformer network.

17. The method of claim 14, further comprising:
determining, using the motion corrected image, one or more anatomical measurements; and
determining a presence or a score of a medical condition, based on the one or more anatomical measurements.

18. The method of claim 17, wherein the motion corrupted image is a high-resolution peripheral quantitative computed tomography (HR-pQCT) image; and
wherein the one or more anatomical measurements include a cortical thickness, a trabecular number, or a bone mineral density; and
wherein the medical condition is osteoporosis.

19. A method comprising:
receiving a plurality of training image pairs, each training image pair of the plurality of training image pairs including:
a first image; and
a second image, wherein the second image is a motion corrupted version of the first image;
providing the plurality of training image pairs to a first machine learning model; and
training the first machine learning model to correct a motion artifact in an image, in response to providing the plurality of training image pairs the first machine learning model.

20. The method of claim 19, further comprising:
receiving a plurality of training images, the plurality of training images including a source image, a motion corrupted version of the source image, and a target image, the source image being substantially free of motion artifacts, the motion corrupted image including an artificially introduced motion artifact, and the target image being a real-world motion artifact image, wherein the source image is of a source domain and the target image is of a target domain;
providing the plurality of training images to a second machine learning model; and
training the second machine learning model to change an inputted image having a source domain into an image having the target domain.
